(19) United States
(12) Patent Application Publication
(10) Pub. No.: US 2026/0233384 A1
(43) Pub. Date: Aug. 13, 2026

(54) SYSTEMS AND METHODS FOR CONTROLLING ROBOTIC MANIPULATORS

(71) Applicant: Bioness Medical, Inc., Valencia, CA (US)

(72) Inventors: Rohit John VARGHESE, Austin, TX (US); Daniel NGUYEN, League City, TX (US); Youngmok YUN, Austin, TX (US)

(21) Appl. No.: 19/470,878

(22) PCT Filed: Apr. 5, 2024
(86) PCT No.: PCT/US2024/023289

§ 371 Date: Sep. 30, 2025

Related U.S. Application Data
(60) Provisional application No. 63/494,894, filed on Apr. 7, 2023.

(51) Int. Cl. B25J 9/10; B25J 9/16

(52) U.S. Cl. CPC B25J 9/1065; B25J 9/1615

(57) ABSTRACT

An embodiment includes systems and methods of creating an equivalent open chain series link representation of closed-loop RCM linkages, for the purpose of simplified inverse dynamics calculations.

### CROSS-REFERENCE TO RELATED APPLICATION

This application claims priority to U.S. Provisional Patent Application No. 63/494,894 filed on Apr. 7, 2023 and entitled “Closed chain serial manipulator dynamics by the use of modified mass and inertia tensors with the Recursive-Newton-Euler formulation”, the content of which is hereby incorporated by reference.

### BACKGROUND

Serial manipulators are an existing class of devices that are used across the industry for a wide range of robotic applications. A manipulator can be thought of as a robotic arm. In order to effectively control these devices, it is important to be able to calculate the forces and torques acting on each joint of the robot due to gravity as well as due to the dynamics of its own movement. Dynamics can be understood as the example of Newton's law “F=ma” where it takes some force to accelerate any mass. So, for the robot to move, especially if that movement is fast, there are many forces due to acceleration. These forces act in each of the three cartesian directions and act as torques in the three rotation directions. When movement across multiple joints is involved, there are additional forces, such as the Coriolis force, that also come into effect.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIGS. 1, 2, and 3 includes systems for implementing methods described herein.

FIGS. 4A-F describe different potential kinematic configurations of an RCM closed chain linkage.

FIG. 5 describes a four-bar linkage and its two force reaction points.

FIG. 6 describes a flowchart of an embodiment of a method for the closed chain RCM to serial cognate link conversion.

FIG. 7 describes a messaging diagram which shows how the cognate link generation process modifies the kinematic and inertial properties of an RCM linkage to an equivalent serial link.

FIG. 8 describes a robotic manipulator comprising an RCM.

FIG. 9 describes a manipulator rigid body table from a MATLAB simulation to model the inverse dynamics of a cognate serial link.

FIG. 10 describes a manipulator rigid body table from a MATLAB simulation to model the true RCM inverse dynamics to compare against the cognate serial link.

FIG. 11 describes a user process flowchart for implementing the proposed cognate serial link in lieu of another closed chain control method.

FIGS. 12A and 12B show an embodiment of an RCM linkage system and why it is useful for exoskeleton applications.

The left portion of FIG. 13 shows a real RCM system similar to FIG. 12A that has the benefits of a remote center. The right portion of FIG. 13 shows a serial “cognate” (meaning equivalent) system.

The mapping of sublink mass and inertia in a real system to cognate system is captured in Table I of FIG. 14.

FIG. 15 includes a mapping of a single RCM mass, mᵢⱼ, to a pre-serial cognate link mass m″ᵢⱼ and serial cognate link mass m′ᵢⱼ.

FIGS. 16A, 16B, and 16C include equations to implement embodiments described herein.

FIG. 17 includes an embodiment of a linkage.

FIG. 18 depicts screw and generalized torque of the cognate system compared against real closed chain linkage.

FIG. 19 depicts screw response comparison between real and cognate system with inertia tensor correction.

FIG. 20 depicts joint torque mismatch between the real and cognate system.

FIG. 21 depicts an actuator torque example when the quasistatic conditions are met.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

Applicant determined the calculation of the above-mentioned forces (generally referred to as computing the inverse dynamics of the system) is very intensive, and analytical equations for even simple manipulators of three joints can be several pages in length, which involves great work in their derivation, with scope for human error, and these must be recalculated for each kind of manipulator. These large equations also require significant computation to execute, thus requiring very powerful computer processor systems to execute them in real time.

There exist conventional techniques to perform these calculations in a recursive loop in a far simplified manner. The Recursive-Newton-Euler (RNE) formulation is a prominent technique commonly used in conjunction with the Denavit-Hartenberg (DH) convention of mathematically describing the manipulator. However, Applicant determined these techniques are all limited to a sub-class of serial manipulators known as open-chain serial manipulators, which are defined as being an open chain of links connected to each other without loops.

Conversely, there are closed chain linkages (which do contain loops) which have inverse dynamics that are more difficult to compute than the serial open chain type. Conventional methods require the user to create custom loop-closure equations for each unique linkage, which is costly in development time and requires a higher degree of expertise from the user.

One class of closed loop linkages are remote center of motion (RCM) mechanisms, which are defined as mechanisms which feature a part which rotates about a fixed point distal to the actuation axis, with no physical revolute joint at the fixed point. These types of linkages are becoming increasingly prevalent in robotics for the purpose of creating virtual joints which move about fixed instant centers (ICs). The use of the recursive Newton Euler formula is prevalent in existing software packages to compute the inverse dynamics for open chains, but Applicant determined these packages are incompatible with current strategies for computing the inverse dynamics of these closed chains. The standard techniques such as the recursive Newton Euler formulation cannot be applied to closed chain manipulators, which greatly reduces the usefulness of these mechanical systems.

In an embodiment, the resulting dynamics calculated for the virtual open chain system are mathematically equal to the actual dynamics of the real closed system. This enables the real-time calculation of dynamics of a specific class of closed chain manipulators in real-time on the processing computer system of the robot.

An embodiment includes a method of creating an equivalent open chain series link representation of closed-loop RCM linkages, for the purpose of simplified inverse dynamics calculations. A given closed loop RCM linkage is algorithmically replaced with a new serial link deliberately specified to imitate the inverse dynamics of the system. The algorithm takes each link mass and inertia in the original closed loop linkage and maps it to the dynamic properties (mass and inertia tensor) of the substituted serial link. By converting closed loop linkages to open chain links, the otherwise difficult inverse dynamics calculations become easily calculated utilizing standard RNE software packages. The inverse dynamics is not perfectly replicated for all systems/conditions, but an embodiment provides error bounds to determine when substitutions are acceptable.

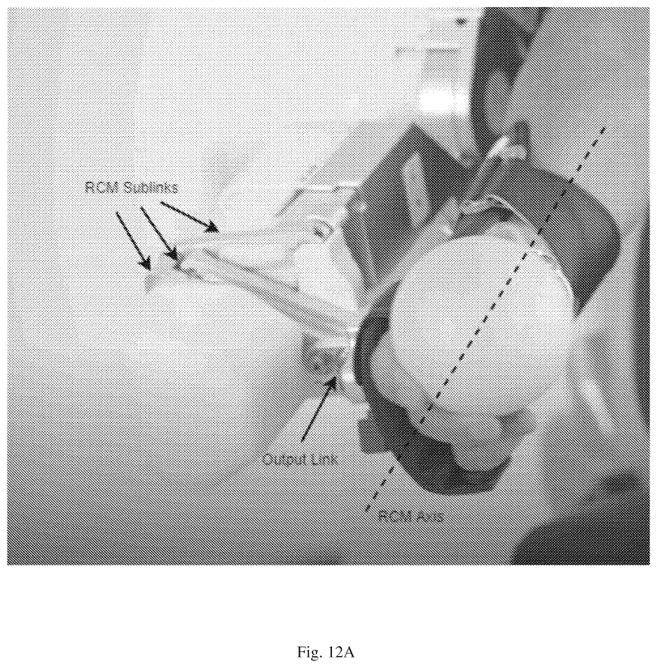

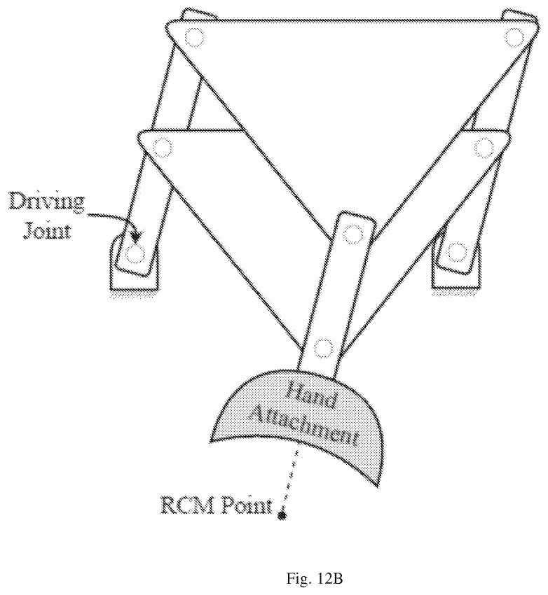

Discussion regarding FIGS. 12A to 21 (see below) provides a more detailed discussion of various embodiments and provides figures/tables related to: (1) an example system of an RCM closed loop linkage attached to a serial chain, (2) an abstracted RCM closed loop linkage, with indexed virtual centers/dynamics properties for each link, (3) screw and generalized torque maps to compare substitution/actual dynamics, and (4) equations for mass and inertia tensor substitution

However, at a higher level of discussion, embodiments enable simplified calculation of the inverse dynamics for robotic systems which are comprised of serial links and RCM closed loop linkages. Embodiments can be utilized to convert the closed loops within the systems into serial links, producing a purely serial chain robot compatible with traditional RNE software packages. In some embodiments there is an associated torque error with converting the closed loop linkage, and embodiments provide guidance on linkage design to minimize the error associated with the conversion.

Embodiments utilize a method of substituting a serial link for a closed loop serial linkage in a software representation of a robot, specifically for the purpose of simplifying the inverse dynamics calculation.

Some embodiments specifically target usage with traditional RNE software for open chains. In conventional systems the kinematic analysis and control are only applicable to rigid control and do not feature any means of force/torque control (necessary in an impedance controller). The inverse dynamics calculation means in some conventional systems require considerable development for constraint forces, linkage modification, etc. and are not compatible with usage in traditional RNE software.

In contrast, embodiments simplify the process of calculating inverse dynamics by making the calculations compatible with RNE software. Conventional methods of calculating inverse dynamics still require custom development of closure/constraint equations, case specific substitutions to convert a closed loop chain to serial link, etc.

Conventional inverse dynamics calculations require the cost of high development time/necessary subject expertise (largely in part since it is incompatible with the popular RNE strategy). In contrast, an embodiment simplifies the process of calculating the inverse dynamics to make it applicable to all RCM closed loop linkages in a general way with less development time necessary.

Embodiments have various applications. For example, embodiments may provide a means of performing inverse dynamics calculations for stroke rehabilitation systems. RCM linkages are utilized in several places in such systems (e.g., Harmony SHR® by Harmonic Bionics) to match anatomical instant centers of motion. Embodiments provide a way to simplify the inverse dynamics calculation of these linkages, which is necessary for impedance control in such systems.

FIGS. 12A and 12B show an embodiment of an RCM linkage system and why it is useful for exoskeleton applications. It is also similarly useful in surgical robotics. The left portion of FIG. 13 shows a real RCM system similar to FIG. 12A that has the benefits of a remote center. However, all methods to solve the dynamics of this system are complex and computationally inefficient. The right portion of FIG. 13 shows a serial “cognate” (meaning equivalent) system that moves in identical/near-identical ways to the RCM system. The serial cognate system can be solved using several widely used techniques, including “recursive-Newton Euler” with has benefits of being practically straightforward to implement and is very computationally efficient.

An embodiment includes the creation of the serial cognate representation in software, creating the equivalent mass and inertial properties that make it dynamically equivalent to the RCM, and solving it using any standard serial manipulator technique.

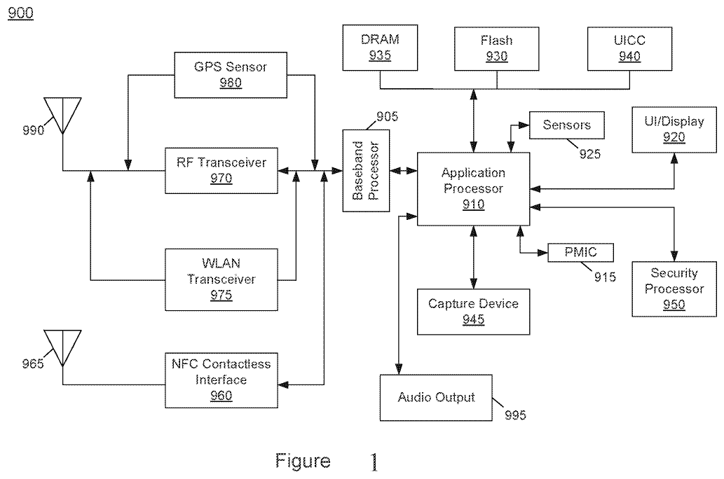

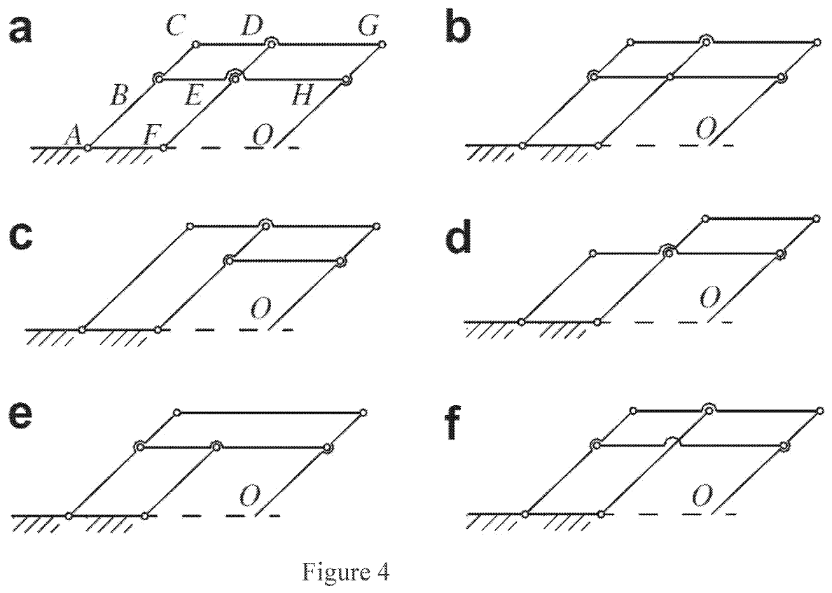

FIGS. 1-3 are addressed below. However, FIGS. 4-11 are first addressed immediately below.

FIG. 4 describes different potential kinematic configurations of an RCM closed chain linkage. Rotating joint nodes are labeled in 4A and each subfigure features different combinations of links and connectivity. All subfigures share a Remote Center of Motion Point (RCMP) O, about which link G-O rotates. The linkage satisfies two criteria: 1) it has a link G-O which rotates about, but does not intersect, the RCMP and 2) there is no physical revolute joint at the RCMP. The RCMP is identical in position across all subfigures but each manifestation of the RCM utilizes different kinematic structures to achieve this RCMP. All of these different linkage structures, by nature of the shared RCM properties, are compatible with the proposed cognate serial link substitution(s).

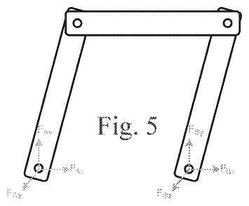

FIG. 5 describes a four-bar linkage and its two force reaction points, at rotational pivots located at the center of attached coordinate Frames A and B. In traditional serial chain linkages, inertial forces and applied external forces/moments result in corresponding reactions at a single pivot. In this embodiment, which is a representative example of a closed chain linkage, the presence of two pivots means that forces/moment reactions are split between the two. Let A be a driven pivot and B be a passive pivot.

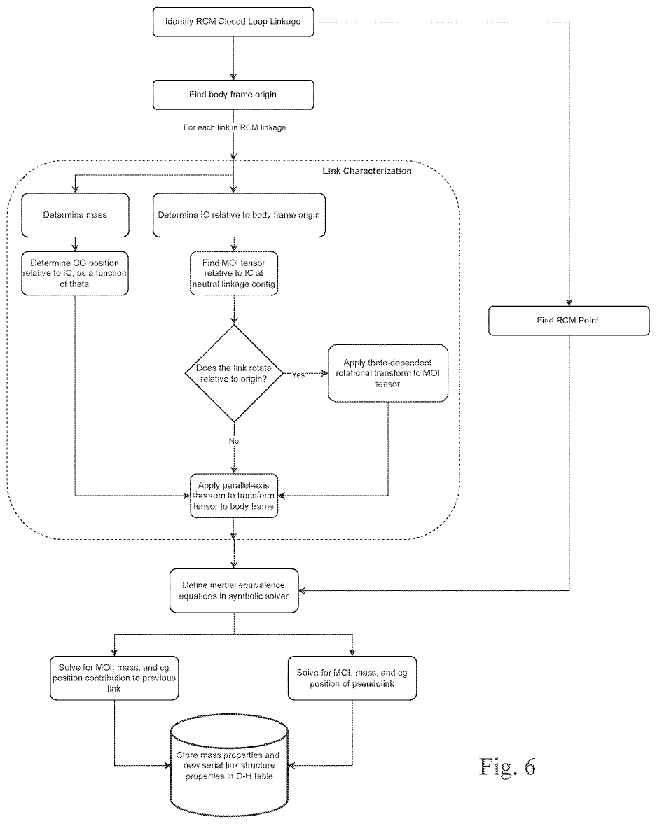

FIG. 6 describes a flowchart of an embodiment of a method for the closed chain RCM to serial cognate link conversion. This process is repeated for each RCM present in a robotic manipulator in order to convert the overall mass matrix of the robot into one consistent with a serial representation. First, a body frame is attached to the RCMP of the RCM with the z-axis coincident with the RCM plane. Properties from each sublink in the RCM are then transformed in the “Link Characterization” stage through several steps to transform their inertial properties to the body frame (originally they are expressed in a sublink frame). This transform is a function of the RCM actuation angle, theta. These contributions from each sublink are summed to form a superposition of the weighted values. Finally, a symbolic solver finds a set of inertial properties of a serial link consistent with the superposition of sublink inertial contributions. These new inertial values are stored in a mass property table in addition to a new kinematic table made with the redefined RCM frames.

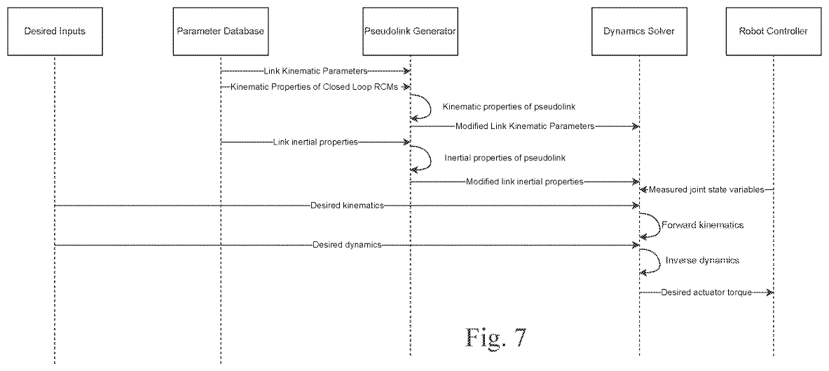

FIG. 7 describes a messaging diagram which shows how the cognate link generation process modifies the kinematic and inertial properties of an RCM linkage to an equivalent serial link. The cognate generator serves as an intermediate step between the parameter database and the dynamics solver. In a traditional Recursive Newton Euler setup for serial links, the cognate generator is not present. Kinematic parameters, kinetic properties of the RCM, and link inertial properties are passed directly from the parameter database to the dynamics solver. In an embodiment, a method adds the cognate generator as an intermediate modifier of all these parameters. The algorithm instead takes these properties from the parameter database and produces a new set of kinematic and inertial properties consistent with a serial link to pass onto the dynamics solver. From there, a standard software package may be utilized for the dynamics solver in which commanded kinematic/dynamic behavior from the input branch, in conjunction with the now modified parameters, are used as a means of calculating desired actuator torques throughout the robot.

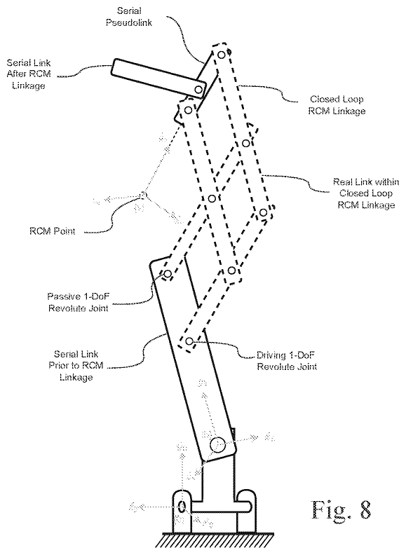

FIG. 8 describes a robotic manipulator comprising an RCM. The RCM is embedded within an otherwise serial structure and its closed chain kinematics make the inverse dynamics of the system unsolvable with standard software packages which require purely serial kinematics. The RCM is positioned between two other serial links via two pivots on each side. Each connection to the prior and following serial links comprises a passive pivot and a driven pivot where an actuator is positioned. The RCM also has the unique property of having the serial cognate link rotate about an instant center labeled the RCM point. The fact that the cognate link does not physically intersect the RCM point and possesses no physical pivot there defines this linkage as an RCM. The serial conversion process removes the entire closed chain RCM linkage and substitutes a single serial link. This cognate serial link is interpreted as being attached to the prior serial link and post serial link, leading to a different inertial representation of the whole manipulator, called the cognate system.

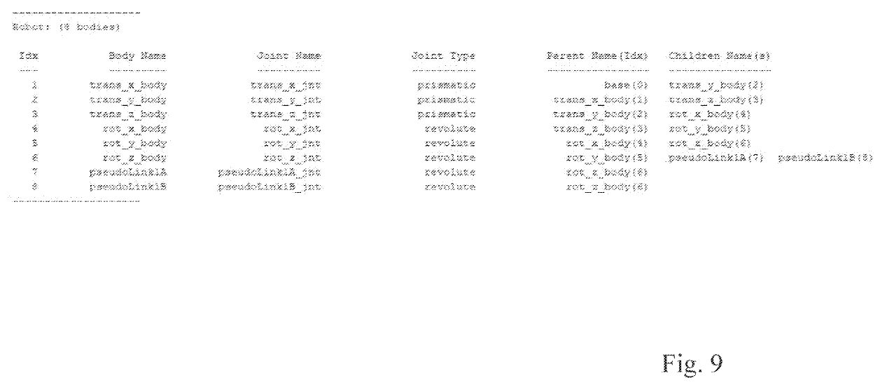

FIG. 9 describes a manipulator rigid body table from a MATLAB simulation to model the inverse dynamics of a cognate serial link. The first 6 rigid bodies correspond to virtual actuators (with no real torque) as a means of applying a spatial velocity/acceleration to the cognate serial link. pseudoLink1A corresponds to a static mass contribution of the cognate link whereas pseudoLink1B corresponds to one dependent on the RCM actuation angle.

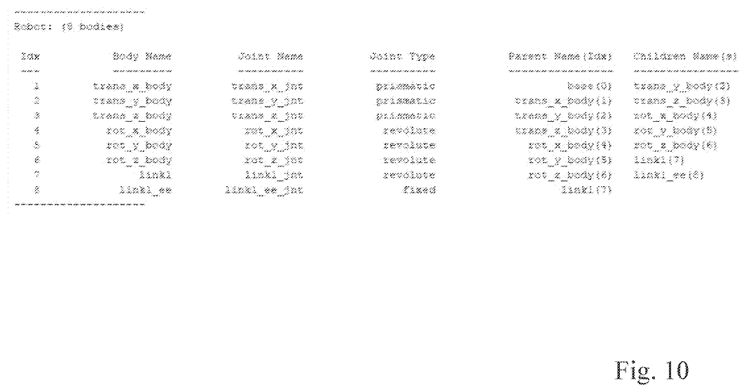

FIG. 10 describes a manipulator rigid body table from a MATLAB simulation to model the true RCM inverse dynamics to compare against the cognate serial link. Similar to FIG. 9, the first 6 rigid bodies are virtual actuators to apply arbitrary spatial velocity/acceleration at the RCMP frame. Link1 represents a sublink of the RCM offset from the RCMP, to test that the inertial mapping of a sublink is consistently captured by the cognate serial link. The inverse dynamics are compared against the serial manipulator of FIG. 9.

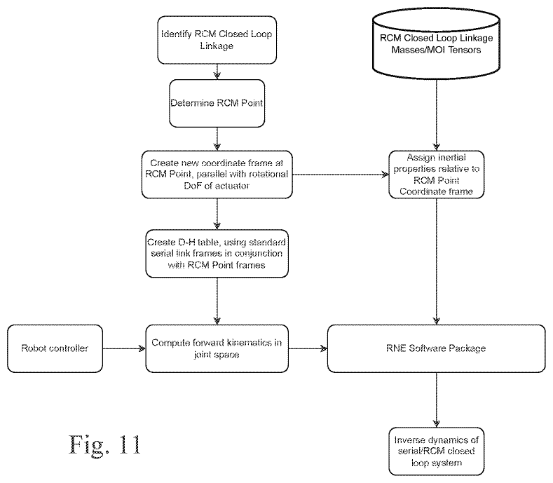

FIG. 11 describes a user process flowchart for implementing the proposed cognate serial link in lieu of another closed chain control method. First, all RCM closed chain linkages in the manipulator are identified and their RCM points are identified. Coordinate frames are positioned at the RCMP which inertial properties are defined relative to in the new cognate serial link. The user then assigns inertial properties to the cognate serial system, utilizing the cognate serial mapping procedure outlined in FIG. 6. A new serial D-H table, utilizing the established RCMP frame, is used as the frame for each cognate serial link in the D-H transforms. The user then utilizes a standard RNE software package with the new D-H and inertial parameters, feeding the inverse dynamics torques to the actuators as in a standard serial system.

FIGS. 1-3 are now addressed.

FIG. 1 includes a block diagram of an example system with which embodiments can be used. As seen, system 900 may be a smartphone or other wireless communicator or any other Internet of Things (IoT) device. A baseband processor 905 is configured to perform various signal processing with regard to communication signals to be transmitted from or received by the system. In turn, baseband processor 905 is coupled to an application processor 910, which may be a main CPU of the system to execute an OS and other system software, in addition to user applications such as many well-known social media and multimedia apps. Application processor 910 may further be configured to perform a variety of other computing operations for the device.

In turn, application processor 910 can couple to a user interface/display 920 (e.g., touch screen display). In addition, application processor 910 may couple to a memory system including a non-volatile memory, namely a flash memory 930 and a system memory, namely a DRAM 935. As further seen, application processor 910 also couples to a capture device 945 such as one or more image capture devices that can record video and/or still images.

A universal integrated circuit card (UICC) 940 comprises a subscriber identity module, which in some embodiments includes a secure storage to store secure user information. System 900 may further include a security processor 950 (e.g., Trusted Platform Module (TPM)) that may couple to application processor 910. A plurality of sensors 925, including one or more multi-axis accelerometers may couple to application processor 910 to enable input of a variety of sensed information such as motion and other environmental information. In addition, one or more authentication devices may be used to receive, for example, user biometric input for use in authentication operations.

As further illustrated, a near field communication (NFC) contactless interface 960 is provided that communicates in a NFC near field via an NFC antenna 965. While separate antennae are shown, understand that in some implementations one antenna or a different set of antennae may be provided to enable various wireless functionalities.

A power management integrated circuit (PMIC) 915 couples to application processor 910 to perform platform level power management. To this end, PMIC 915 may issue power management requests to application processor 910 to enter certain low power states as desired. Furthermore, based on platform constraints, PMIC 915 may also control the power level of other components of system 900.

To enable communications to be transmitted and received such as in one or more internet of things (IoT) networks, various circuits may be coupled between baseband processor 905 and antenna 990. Specifically, a radio frequency (RF) transceiver 970 and a wireless local area network (WLAN) transceiver 975 may be present. In general, RF transceiver 970 may be used to receive and transmit wireless data and calls according to a given wireless communication protocol such as 5G wireless communication protocol such as in accordance with a code division multiple access (CDMA), global system for mobile communication (GSM), long term evolution (LTE) or other protocol. In addition, a GPS sensor 980 may be present, with location information being provided to security processor 950. Other wireless communications such as receipt or transmission of radio signals (e.g., AM/FM) and other signals may also be provided. In addition, via WLAN transceiver 975, local wireless communications, such as according to a Bluetooth™ or IEEE 802.11 standard can also be realized.

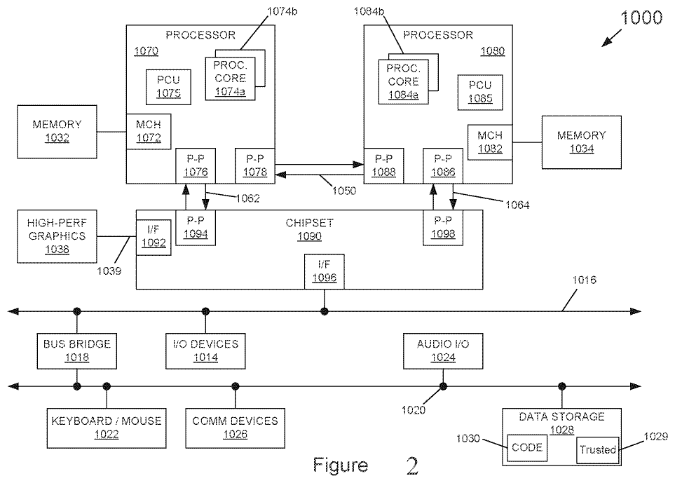

FIG. 2 shows a block diagram of a system in accordance with another embodiment of the present invention. Multiprocessor system 1000 is a point-to-point interconnect system such as a server system, and includes a first processor 1070 and a second processor 1080 coupled via a point-to-point interconnect 1050. Each of processors 1070 and 1080 may be multicore processors such as SoCs, including first and second processor cores (i.e., processor cores 1074a and 1074b and processor cores 1084a and 1084b), although potentially many more cores may be present in the processors. In addition, processors 1070 and 1080 each may include power controller unit 1075 and 1085. In addition, processors 1070 and 1080 each may include a secure engine to perform security operations such as attestations, IoT network onboarding or so forth.

First processor 1070 further includes a memory controller hub (MCH) 1072 and point-to-point (P-P) interfaces 1076 and 1078. Similarly, second processor 1080 includes a MCH 1082 and P-P interfaces 1086 and 1088. MCH's 1072 and 1082 couple the processors to respective memories, namely a memory 1032 and a memory 1034, which may be portions of main memory (e.g., a DRAM) locally attached to the respective processors. First processor 1070 and second processor 1080 may be coupled to a chipset 1090 via P-P interconnects 1062 and 1064, respectively. Chipset 1090 includes P-P interfaces 1094 and 1098.

Furthermore, chipset 1090 includes an interface 1092 to couple chipset 1090 with a high-performance graphics engine 1038, by a P-P interconnect 1039. In turn, chipset 1090 may be coupled to a first bus 1016 via an interface 1096. Various input/output (I/O) devices 1014 may be coupled to first bus 1016, along with a bus bridge 1018 which couples first bus 1016 to a second bus 1020. Various devices may be coupled to second bus 1020 including, for example, a keyboard/mouse 1022, communication devices 1026 and a data storage unit 1028 such as a non-volatile storage or other mass storage device. As seen, data storage unit 1028 may include code 1030, in one embodiment. As further seen, data storage unit 1028 also includes a trusted storage 1029 to store sensitive information to be protected. Further, an audio I/O 1024 may be coupled to second bus 1020.

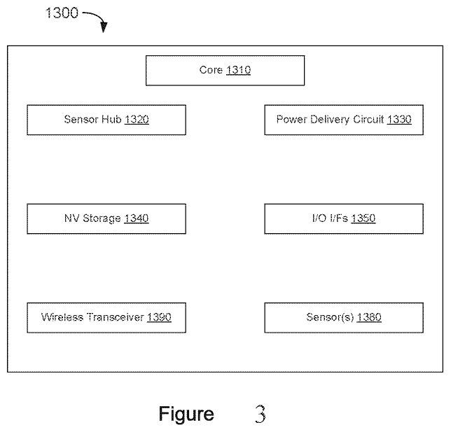

FIG. 3 depicts an IoT environment that may include wearable devices or other small form factor IoT devices. In one particular implementation, wearable module 1300 may be an Intel® Curie™ module that includes multiple components adapted within a single small module that can be implemented as all or part of a wearable device. As seen, module 1300 includes a core 1310 (of course in other embodiments more than one core may be present). Such a core may be a relatively low complexity in-order core, such as based on an Intel Architecture® Quark™ design. In some embodiments, core 1310 may implement a Trusted Execution Environment (TEE). Core 1310 couples to various components including a sensor hub 1320, which may be configured to interact with a plurality of sensors 1380, such as one or more biometric, motion, environmental or other sensors. A power delivery circuit 1330 is present, along with a non-volatile storage 1340. In an embodiment, this circuit may include a rechargeable battery and a recharging circuit, which may in one embodiment receive charging power wirelessly. One or more input/output (IO) interfaces 1350, such as one or more interfaces compatible with one or more of USB/SPI/I2C/GPIO protocols, may be present. In addition, a wireless transceiver 1390, which may be a Bluetooth™ low energy or other short-range wireless transceiver is present to enable wireless communications as described herein. In different implementations a wearable module can take many other forms. Wearable and/or IoT devices have, in comparison with a typical general-purpose CPU or a GPU, a small form factor, low power requirements, limited instruction sets, relatively slow computation throughput, or any of the above.

Embodiments may be used in many different types of systems. For example, in one embodiment a communication device can be arranged to perform the various methods and techniques described herein. Of course, the scope of the present invention is not limited to a communication device, and instead other embodiments can be directed to other types of apparatus for processing instructions, or one or more machine readable media including instructions that in response to being executed on a computing device, cause the device to carry out one or more of the methods and techniques described herein.

Program instructions may be used to cause a general-purpose or special-purpose processing system that is programmed with the instructions to perform the operations described herein. Alternatively, the operations may be performed by specific hardware components that contain hardwired logic for performing the operations, or by any combination of programmed computer components and custom hardware components. The methods described herein may be provided as (a) a computer program product that may include one or more machine readable media having stored thereon instructions that may be used to program a processing system or other electronic device to perform the methods or (b) at least one storage medium having instructions stored thereon for causing a system to perform the methods. The term “machine readable medium” or “storage medium” used herein shall include any medium that is capable of storing or encoding a sequence of instructions (transitory media, including signals, or non-transitory media) for execution by the machine and that cause the machine to perform any one of the methods described herein. The term “machine readable medium” or “storage medium” shall accordingly include, but not be limited to, memories such as solid-state memories, optical and magnetic disks, read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, as well as more exotic mediums such as machine-accessible biological state preserving or signal preserving storage. A medium may include any mechanism for storing, transmitting, or receiving information in a form readable by a machine, and the medium may include a medium through which the program code may pass, such as antennas, optical fibers, communications interfaces, and the like. Program code may be transmitted in the form of packets, serial data, parallel data, and the like, and may be used in a compressed or encrypted format. Furthermore, it is common in the art to speak of software, in one form or another (e.g., program, procedure, process, application, module, logic, and so on) as taking an action or causing a result. Such expressions are merely a shorthand way of stating that the execution of the software by a processing system causes the processor to perform an action or produce a result.

A module as used herein refers to any hardware, software, firmware, or a combination thereof. Often module boundaries that are illustrated as separate commonly vary and potentially overlap. For example, a first and a second module may share hardware, software, firmware, or a combination thereof, while potentially retaining some independent hardware, software, or firmware. In one embodiment, use of the term logic includes hardware, such as transistors, registers, or other hardware, such as programmable logic devices. However, in another embodiment, logic also includes software or code integrated with hardware, such as firmware or micro-code.

FIGS. 12A-23 are now addressed.

Embodiments include a generalized solution for the inverse dynamics of closed chain remote center of motion linkages, enabling their use with standard open chain software packages. Remote center of motion linkages are being increasingly utilized in robotic exoskeletons and medical robotics. Current techniques for the calculation of inverse dynamics of these closed chain linkages are non-generalized and require time consuming development of custom loop closure equations, which are obstacles to the development of real-time control strategies. The computation of inverse dynamics for serial kinematic chains, in contrast, is well established and simplified by utilizing common and efficient recursive Newton-Euler software packages. An embodiment uses a method of kinetic cognates to convert the closed chain to an equivalent serial representation which is not only compatible with standardized solvers but also eliminates redundant computation corresponding to passively driven links, reducing the numerical complexity of the recursive Newton-Euler process from O(N) to O(N′), where N—N′ is the number of passive links in the closed chain system. In summary, embodiments include a method of kinetic cognates that reduces development time by enabling the use of existing recursive Newton Euler software packages, improves computational efficiency, and has the potential applications to a range of closed chain mechanisms.

### I. Introduction

Historical developments of inverse dynamics methods have been motivated by a need for algorithms fast enough to execute real-time control. Serial manipulators in particular were emphasized in the first inverse dynamics formulations and the Recursive Newton-Euler (RNE) algorithm established itself as the de-facto standard for solving real-time inverse dynamics problems [1], [2]. Modern implementation of inverse dynamics often relies on shared software packages implementing RNE, but these existing packages [3]-[5] are primarily intended for use with serial kinematic chains and incompatible with closed chain topologies. Closed chains are becoming more prevalent in robotic design due to the ability to generate workspace constraints mechanically, but there lacks a generalized and efficient method such as RNE for computing the inverse dynamics of these structures.

### A. Remote Center of Motion Linkages

A growing class of closed chain linkages are remote center of motion (RCM) mechanisms, which are becoming increasingly prevalent in medical robotics [8]-[11] for the purpose of creating virtual joints which move about fixed instant centers (ICs). Some common use cases include creating a fixed point of entry for minimally invasive surgery or matching anatomical ICs for exoskeletons. Each of these closed chain RCM mechanisms features a link which rotate about a fixed remote center of motion point (RCMP) with respect to the body frame, yet does not possess a physical revolute joint at that location. FIG. 12B provides an example of RCM Mechanism, where the output link is constrained to rotate about the RCMP O by nature of the parallelogram structure.

[[FIG. 12A: The Harmony SHR® [6], [7] wrist pronation linkage (a) is an example of the practical utility of a remote center of motion (RCM) mechanism for physical human-robot interaction. Rotation of the wrist occurs about a fixed pronation axis and the creation of an RCM axis via linkage geometry permits the actuator to be located on a different axis. The addition of a closed chain RCM linkage allows this anatomical motion to be matched without creating a more complex serial arrangement. A planar view schematic of the linkage is presented in FIG. 12B.]]

One example of the practical utility of an RCM mechanism can be found on Harmony SHR® [6], [7], an exoskeleton for neurorehabilitation. As shown in FIG. 12A, an RCMP is designed to be coincident with the wrist pronation axis, circumventing the need to place an actuator coaxially to the forearm. This freedom in placement permits the actuator to be located at a position where it does not interfere with access to the patient forearm (a key requirement for the neurorehabilitation exoskeleton) and reduces the amount of distal mass distribution. The closed chain design solves a key kinematic and spatial requirement of the system, but complicates the process of solving for the inverse dynamics.

### B. Current Methods for Closed Chain Inverse Dynamics

The current challenges of the inverse dynamics problem for closed chains may be divided into two categories: practical and computational. Some recent approaches have attempted to improve on computation time by implementing Newton-Euler and Lagrangian techniques with varying constraint equation representations [12]-[14]. Others have attempted to develop coordinate systems and corresponding generalized inertia representations [15], to reduce discretization error. These approaches are computationally sufficient to execute real-time control without significant error, but require re-derivation of the equations of motion for any changes in the kinematic structure. This is a hindrance in the design of new RCM linkages. Conversely, the computational cost of state-of-the-art constraint equations is prohibitive for real-time control of complex linkages where expressing the numerous constraints becomes numerically challenging.

In contrast to the widely adopted RNE algorithm for serial manipulators, modern methods for computing the inverse dynamics of closed chain manipulators are varied in formalism (Newton-Euler, Lagrangian, Kane), structure (closed-form, recursive), and mathematical representation of physical quantities (Cartesian-Tensor, Spatial Coordinates, Natural Coordinates). Closed chain inverse dynamics are difficult compute due to the requirement that joint variables must satisfy complex loop-closure constraint conditions. State-of-the-art methods differ primarily in how these constraint conditions are expressed numerically and solved for in real-time control loops [12], [17]. A common challenge in these approaches is the need to develop either custom governing equations or software constraints that complicate the process of designing the linkage and then integrating its physical parameters into a control loop.

### C. Serial Kinetic Cognates of Closed Chain RCMs

Historically, RNE emerged due to the inability to compute inverse dynamics for real-time control since the prior algorithms such as Uicker-Khan were not computationally efficient enough. Analyses of the advantages of RNE demonstrated that its recursive nature and mathematical representations of physical quantities, not the formalism itself, were responsible for the efficiency improvements [1], [2]. Although modern RNE packages have made the inverse dynamics problem for serial chains simple to formulate and solve, closed chain inverse dynamics implementation still faces computational and practical difficulty. Similar problems existed in the development history for serial chain inverse dynamics, and prior to the adoption of RNE, approximations of the equations of motion such as ignoring Coriolis terms were proposed to improve slower algorithms [19].

Embodiments provide a somewhat analogous simplification method to solve the inverse dynamics for the increasingly popular RCM class of closed chain linkages. First, a joint-space-inertia equivalent serial representation of the closed chain is produced through solving inertial-equivalency statements between the real closed chain RCM system and a fictitious “serial cognate system”. These result in inertial properties which are in the form of a serial chain. For each closed chain RCM linkage, an equivalent serial chain is substituted comprising a “serial cognate link” constrained to rotate about the RCMP of the real RCM mechanism and a “pre-serial cognate link”. A recursive process of replacing each RCM mechanism in a kinematic chain with serial cognate links and pre-serial cognate links creates a dynamically equivalent serial system. This equivalent serial cognate system is directly implementable into RNE packages to accurately capture the inverse dynamics of closed chain systems in a generalized strategy for all RCMs, leveraging the computational advantages and widespread availability of RNE. A solver is provided to the user which takes the kinematic and inertial properties of an RCM as input and returns a serial cognate system implementable into RNE packages which produces equivalent inverse dynamics.

Embodiments provide an algorithmic process for converting a 1-degree of freedom (DoF) planar RCM into an equivalent serial representation. Section II describes a mathematical framework for representing the RCM as a series of moving masses and inertia transforms, parameterized by the actuation angle of the active link. Section IV returns to the motivating example in FIG. 13 and solves for its equivalent serial representation via an embodiment. Section discuss possible limitations of the approach and the results from the case study. Section VI provides a conclusion.

### II. Methodology

This section establishes an algorithmic process for converting a 1-DoF planar RCM into an equivalent serial representation. The section describes a mathematical frame-work to parameterize the kinematics of each sublink within an RCM linkage. The section then demonstrates how the unique kinematic constraints of a planar RCM allow equivalency statements for inverse dynamics to be created between the original system and a proposed serial cognate system. The equivalency system of equations is then solved to produce the inertial properties for this equivalent cognate system.

### A. Equivalent Series Link Replacement Framework

[[FIG. 13: Representation of a closed chain RCM linkage embedded within a serial kinematic chain. Frame 0 is an inertial stationary world frame whereas Frames i are associated with Links i. Link 3, in the real system, is constrained to rotate about the RCM coincident with Frame 3. Unlike the other links, it does not possess a physical revolute joint at its instant center, but is instead constrained to rotate about the RCMP by the RCM sublinks. Because Link 3 kinematically behaves equivalently to a serial link, it can be treated as such in an RNE implementation.]]

A system containing a closed chain RCM system is depicted in FIG. 13, where blue RCM sublinks comprise the RCM linkage. These sublinks constrain the output Link 3 to rotate about an RCMP. The closed chain RCM linkage system may be converted to an equivalent serial system by defining a method of mapping the real RCM sublink inertial properties to those belonging to serial rigid bodies in the cognate system. In this case, Links 2′ and 3′, termed the pre-serial cognate link and serial cognate link respectively, have masses which are dependent on the sublink masses.

The mapping of each sublink mass and inertia in the real system to the cognate system is captured in Table I (FIG. 14), with the colors of the mass in the table corresponding to those depicted in FIG. 2. The real RCM sublinks influence the properties of Link 2′ and Link 3′, termed the pre-serial cognate link and serial cognate link respectively.

For a serial chain of n links and reference frames, let i represent an iterator for each reference frame (with Link i corresponding to Frame i). For instances where a frame is associated with an RCM, an additional index j is introduced to iterate over the sublinks. The reference coordinate frame for the RCM is taken to be centered at the RCMP. An example of this indexing is shown in FIG. 13. Table I (FIG. 14) demonstrates how the sublink masses m₃ⱼ from the real system end up contributing to the Link 2′ and 3′ masses. This solely serves to demonstrate qualitative dependencies, and the actual equations behind this mapping are explored in Section II. Notably, the real sublinks cannot be used in an RNE implementation since there is not a reference frame for every sublink-only at the RCMP. By mapping these masses and inertias to masses m′ and m′3 which correspond to frames of rigid body links in the cognate system, the system simplifies to one which is compatible with RNE. Table I shows how all sublinks are eliminated in the cognate system.

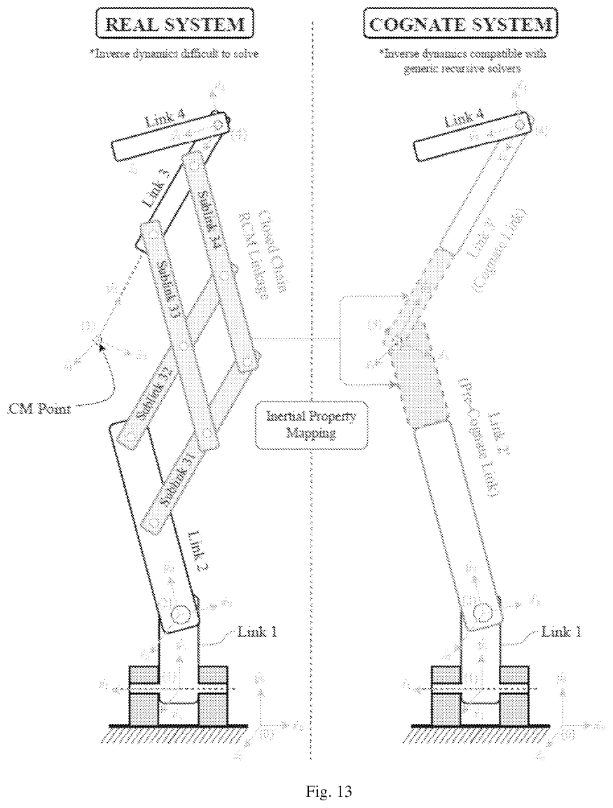

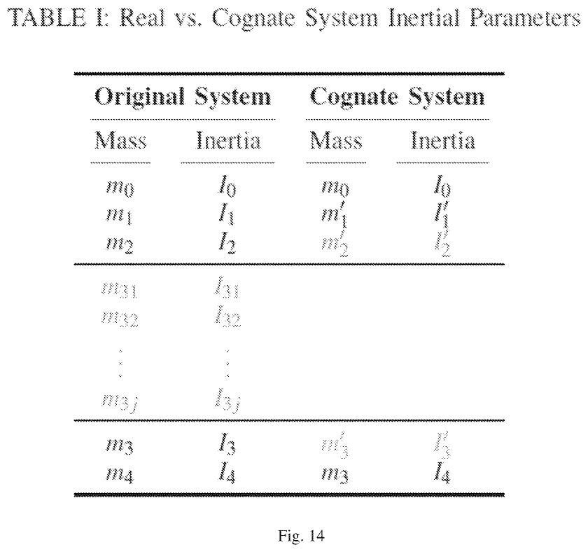

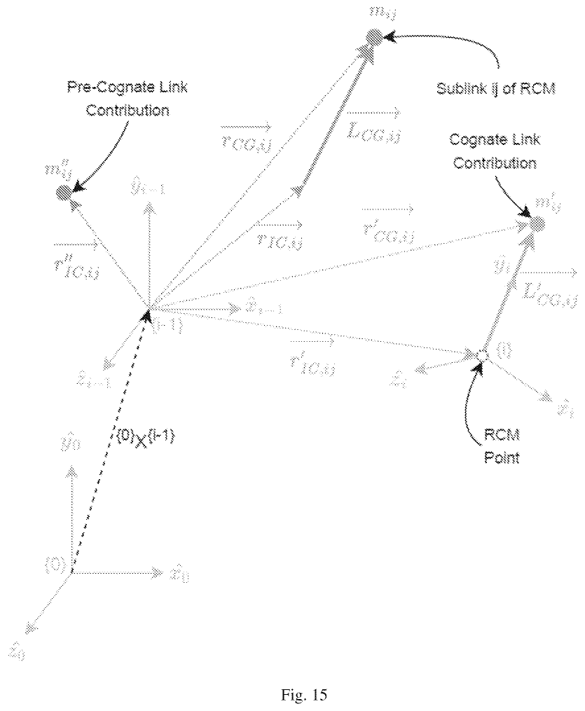

The following RCM mechanism properties must exist to produce equivalent series representations using some embodiments described herein:
1) The closed chain linkage is 1-DoF and planar (all joints are 1-DoF revolute, with the rotation axes all parallel).
2) The center of gravity (CG) of any given sublink must move about a fixed instant center with respect to the body frame of the linkage.
3) Only two rotational velocities exist for all links in the linkage with respect to the linkage body frame (zero, and the angular velocity of the driven joint).
4) The output coordinate frame of the RCM linkage must be located at the RCMP.
5) The number of active joints equals the DoF of the system (it is neither underactuated nor overactuated).

[[FIG. 15: Mapping of a single RCM mass, mᵢⱼ, to the pre-serial cognate link mass m″ᵢⱼ and serial cognate link mass m′ᵢⱼ. Each of the mᵢⱼ entries corresponds to the blue sublinks in the example closed chain RCM mechanism of FIG. 2. m″ᵢⱼ and m′ᵢⱼ similarly correspond to Link 2′ and Link 3′ (the pre-pseudolink and serial cognate link). The pre-serial cognate link contribution m″ᵢⱼ and serial cognate link contribution m′ᵢⱼ are computed for each sublink, and weighted sum equations in Eq. 7-11 give the mass and position of the new serial cognate links.]]

The kinematics of the RCM mechanism defined in FIG. 15 may be broken down into the motions of each of the links: each of these links rotates about a fixed IC and contributes differently to the inverse dynamics of the system. Frame 0 denotes an inertial world frame and Frame i denotes the non-inertial body frame of a closed chain linkage. Note that since Frame i rotates with each sublink, the rotations are described relative to Frame i-1. Frame i-1 has a displacement transform ⁰X^{i-j} relative to the world frame and associated kinematic values of

$${\to }_{{v}_{b}} , {\to }_{{ω}_{b}} , {\to }_{{a}_{b}} , and {\to }_{{α}_{b}}$$

which can be found using the Jacobian of joint angles to cartesian coordinates leading up to the frame. mᵢ represents the mass (at the CG) of one of the links within the closed chain structure, which is constrained to rotate about an

$$IC {\to }_{{r}_{IC , i , j}}$$

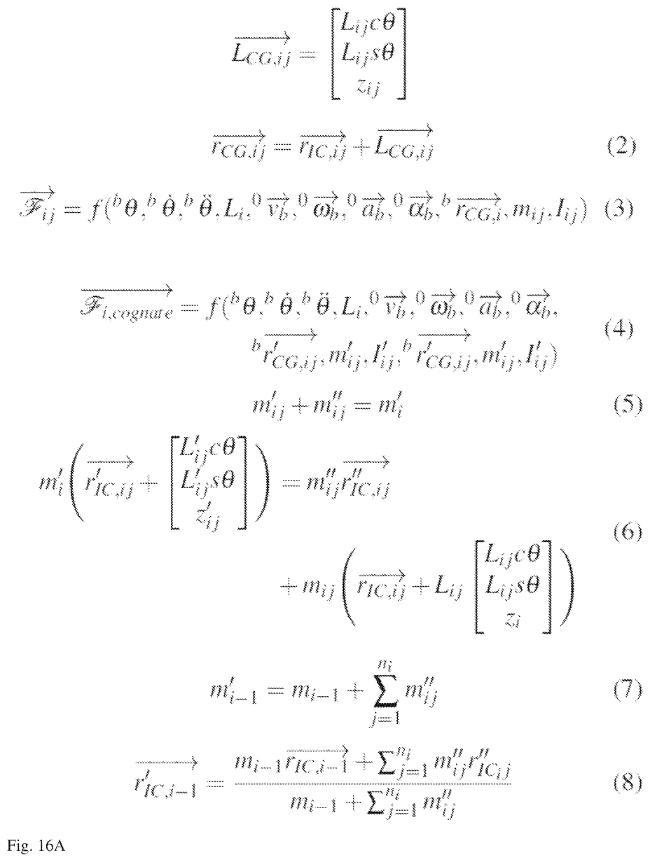

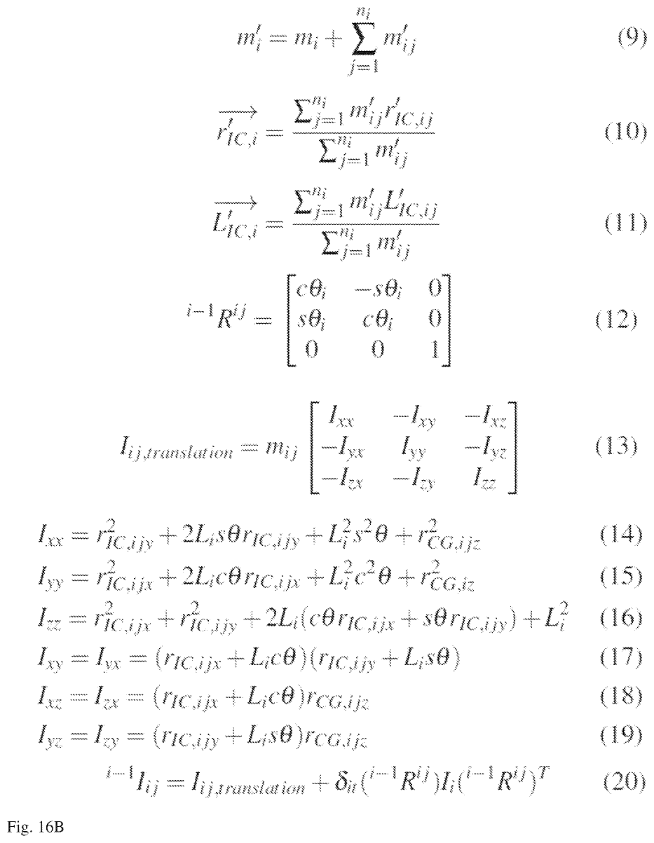

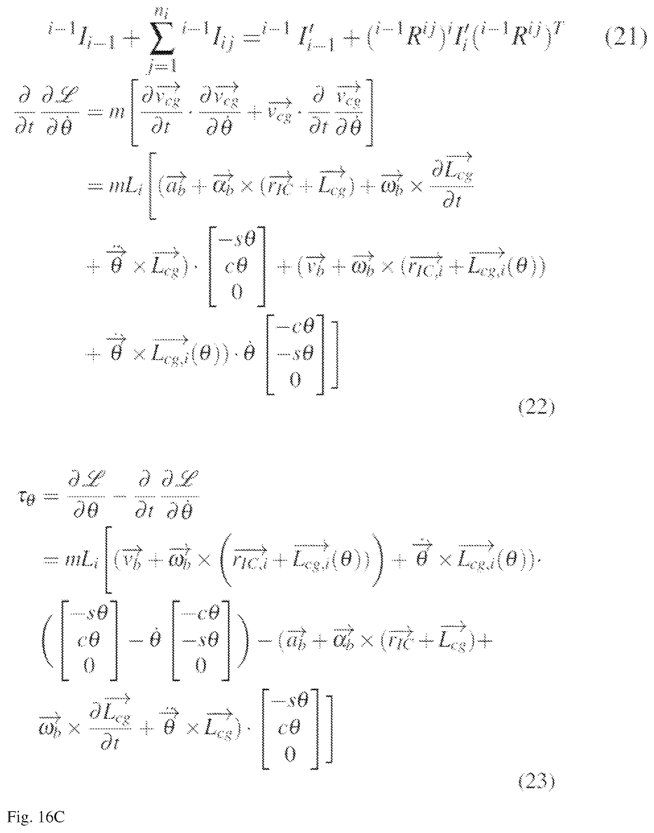

with the CG position relative to the IC described by Eq. 1 (FIG. 16A). The displacement vector from Frame i-1 to mᵢⱼ is then given by Eq. 2 (FIG. 16A). Sublink ij additionally has an associated inertia tensor Iᵢⱼ.

To construct the equivalent series link system, each of the masses mᵢⱼ and inertias Iᵢⱼ are mapped to a serial cognate link and pre-serial cognate link as demonstrated in FIG. 13. These are denoted mass m′ᵢⱼ and cognate inertia m″ᵢⱼ respectively. Each sublink contributes a mass and inertia to the serial cognate link and pre-serial cognate link. A weighted sum of these contributions is then carried out to procedurally generate the mass position and inertial properties of the new system. m′ᵢⱼ is applied to the previous rigid body in the robotic structure (and hence rotates with Frame i-1) and m″ᵢⱼ revolves about the RCMP at Frame i. Two new inertia tensors I′ᵢⱼ and I″ᵢⱼ are also mapped. This process is carried out recursively for each sublink in the real closed chain system, until a collection of masses and inertias are applied to serial links i and i-1 in the cognate system.

### B. CG Jacobian Equivalency

To imitate the inverse dynamics of the closed chain properly, the screw

$${\to }_{{F}_{ij}}$$

applied by any sublink ij in the closed chain to Frame i must be equivalent to the screw

$${\to }_{{F}_{ij , cognate}}$$

which arises from cognate masses m′ᵢⱼ, m″ᵢⱼ and cognate inertias I′ᵢⱼ, and I″ᵢⱼ in the cognate systems.

$${\to }_{{F}_{ij}}$$

is dependent on the forward kinematics of the system and the dynamic properties of the link, as shown in Eq. 3 (FIG. 16A) and 4 (FIG. 16A).

First, an embodiment includes a strategy to solely match the force components of the new cognate wrench

$${\to }_{{F}_{ij , cognate}} .$$

The force component only depends on the forward kinematics of the center of gravity. In other words, if the CG Jacobians of the real and cognate systems match, then the force components of the wrench will be the same [20]. If the total mass of both systems is also the same, then the force reactions must be equivalent. Eqs. 5 (FIG. 16A) and 6 (FIG. 16A) are constraints for each sublink mapping which enforce these properties on the system.

$${\to }_{{r}_{IC , i , j}^{′}}$$

is automatically constrained to De at the RCMP of the linkage. After assigning all known instant centers rIC,ij and masses of the sublinks, 6 unknowns pertaining to the cognate system remain: m′ᵢⱼ, m″ᵢⱼ,

$${\to }_{{r}_{IC , i , j}^{″}} ,$$

and L′ᵢⱼ. Eq. 6 (FIG. 16A) enforces 5 constraints per sublink, since the x and y components of the vectorization each have θ dependent and independent terms which must be satisfied. The nonlinear system of 6 equations can be solved to determine the mass mapping from the real to cognate system. The resultant pre-serial cognate link has a mass given by Eq. 7 (FIG. 16A) and a position relative to Frame i-1 given by Eq. 8 (FIG. 16A). The pre-serial cognate link is a superposition of the real link and the sublink contributions, which is reflected in the weighted sums. The mass and position mapping to the serial cognate link are similarly given by Eq. 9 (FIG. 16B) and 10 (FIG. 16B). Since the serial cognate link rotates about the RCMP, it possesses an additional sum in Eq. 11 (FIG. 16B) for the rotation of each CG contribution about the RCMP. This process can be visualized as taking each of single sublink mappings in FIG. 15 and carrying out a weighted sum to “construct” a new pre-serial cognate link and serial cognate link.

### C. Inertia Tensor Equivalency

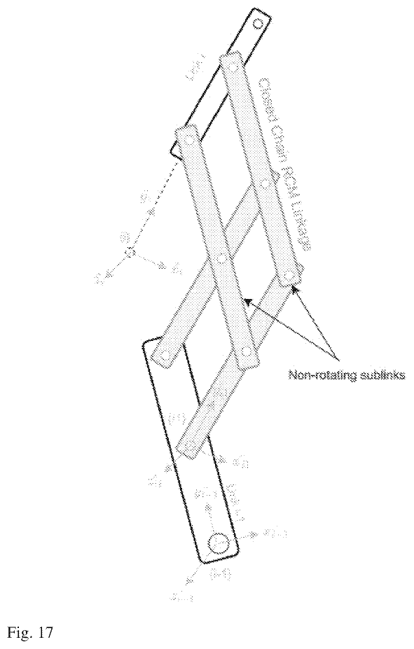

To match the torque components of the screw for the cognate links, additional corrections in the form of inertia tensors I′ᵢ₋₁ and I′ᵢ must be made. By matching the inertia tensor of the cognate serial link to that of the RCM linkage in Frame i-1, the torque component of the screw in that frame will be matched as well. This is because the rotational Jacobian up to Frame i-1 is equivalent in both representations by virtue of keeping Frame i-1 and Frame i in the same positions. Thus, the total closed chain linkage inertia in Frame i-1 uniquely defines the torque components of the screw. The first step is to express each link inertia in Frame i-1. If ^{ij}Iᵢⱼ, the inertia tensor for each sublink, is defined about each sublink coordinate frame as demonstrated in FIG. 17, it rotates by angle θᵢ as the closed chain is actuated. However, some sublinks such as sublinks 33 and 34 in FIG. 13 do not rotate as their CG translates about the instant center. Let these sublinks have sublink index t. Thus, the conversion to inertia in Frame i-1 may be performed by:
1) A similarity transform rotates ^{ij}Iᵢⱼ (inertia tensor of sublink ij in Frame ij) to be aligned with Frame i-1, excluding non-rotating links t.
2) The parallel axis theorem is used to modify the now rotationally aligned sublink inertia matrices such that they are positionally coincident with Frame i-1.

The total resultant transform of the sublink inertia tensors from Frame ij to Frame i-1 is expressed in Eq. 12-20 (FIG. 16B).

There are two sources of θ dependency that result from the overall transformation. The rotational alignment portion of Eq. 20 (FIG. 16B) depends explicitly on sinusoidal functions of θ. In contrast, the planar motion of the CG is encapsulated in the translation tensor of Eq. 13 (FIG. 16B). Similarly to the CG Jacobian strategy, the inertia matrices of the serial cognate link and pre-serial cognate link must be modified to have an equivalent inertia tensor to the total real inertia in Frame i-1.

The system in Eq. 21 (FIG. 16C) may be solved for ⁱ⁻¹1I′ᵢ₋₁ and ⁱI′_{I} in the cognate system. Since the form of a given inertia tensor has 6 unique terms, there are 12 additional constraints created by this new assertion (each term contributes 2 due to the separation of θ terms from each sublink contribution). There are simultaneously 12 additional degrees of freedom introduced as we seek to define I′ᵢ₋₁ and I′ᵢ. Thus, there still remains a unique solution to the link substitution problem.

### D. Generalized Torque Equivalency

During the inverse dynamics backwards iterations in RNE, the wrench at each link is computed by summing the wrench contributions from all the distal links given twists and externally applied wrenches to the system. In a typical series linkage, the component of the screw which aligns with the actuator axis for a given link is taken to be the joint torque required [21]. However, closed chain linkages introduce an additional challenge since the z component of the total wrench on the linkage does not necessarily equate to the actuator torque [22].

In FIG. 13, sublinks 31 and 32 both serve as the physical connection points from Frame 2 to 2. Unlike serial links, there are two points of attachment to the previous frame. The wrench will be given about the virtual actuator at the RCMP at Frame 3. An issue is that a torque (either part of an external wrench or inertial reactions) can potentially be reacted by a force couple at the two physical connection points. In a typical serial link, the only means of reacting the z-component of the torque is via the actuator itself and a component of the wrench provided by RNE may simply be taken as the necessary actuator effort. However, this correlation does not hold for closed chains.

Thus, a small error in the torque arises if the z component of the cognate link wrench is simply taken to equal the actuator torque required. The true torque of the RCM is shown in Eq. 22 (FIG. 16C), 23 (FIG. 16C), where a Lagrangian is solved for the RCM system with the generalized coordinate being the RCM actuation angle, to determine the generalized torque required.

Although the actuator torque is not currently matched by the cognate link in all dynamic cases, the torques are matched in a specific quasi-static case:

$${\to }_{{α}_{b}} = {\to }_{{ω}_{b}} = 0.$$

### III. Case Study

The MATLAB Robotics System Toolbox [4] was utilized to validate that the true screw and cognate screw match for the structure shown in FIG. 17. Since the toolbox does not support closed chains, additional 1-DoF joints for each sublink were created and forced to have the same rotation angle (inefficient in practice, but required for this comparison).

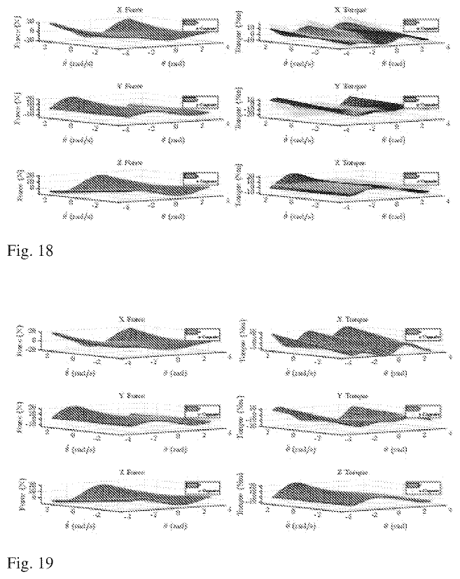

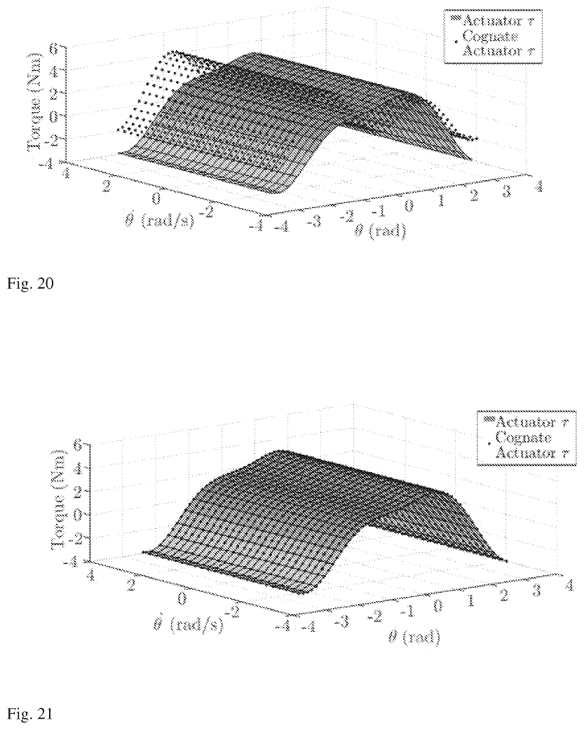

The plots are presented in incremental order of the system of equations featured in Section II-B, II-C, and II-D. FIG. 18 displays a plot of the wrench applied to the system about Frame i-1 as a function of θ and \dot{θ}, with non-zero velocities and accelerations applied to Frame i-1. This is prior to any corrections for the inertia tensor of the cognate system. FIG. 19 introduces the equations featured in Section IIC to further constrain the cognate system to properly match the complete wrench. Finally, FIG. 20 and FIG. 21 demonstrate the Section IID discussion on the fact that the RCM actuator torque is not properly captured, in spite of the wrench about Frame i-1 being properly represented by the cognate system.

[[FIG. 17: Simple example of an RCM grafted from FIG. 13. The inverse dynamics for this case example are expressed about Frame i (which is fixed to Link 1), with prescribed cartesian velocities and accelerations of the coordinate frame to capture arbitrary forward kinematics from a robot with prior Frames 0→i-1.]]

[[FIG. 18: Screw and generalized torque of the cognate system compared against real closed chain linkage.

$$0 {\to }_{{v}_{b}} , 0 {\to }_{{ω}_{b}} , {\to }_{{a}_{b}} , and {\to }_{{α}_{b}}$$

are set to non-zero values and the surface denote the force/torque required to keep \dot{θ}=0. The torque components of the screw do not match with the cognate system mappings defined thus far.]]

[[FIG. 19: Screw response comparison between real and cognate system with inertia tensor correction. By introducing modifications to the serial cognate link and pre-serial cognate link inertial matrices, the mismatch of the torque components in FIG. 18 is eliminated and proper response is achieved in the torque components.]]

[[FIG. 20: Joint torque mismatch between the real and cognate system. The mismatch occurs since the z-component of the wrench torque is not fully reacted by the cognate actuator motor.]]

[[FIG. 21: Actuator torque example when the quasistatic conditions are met. The actual RCM torque and torque of the serial cognate link coincide.]]

### IV. Results

The conversion of the original RCM system to an equivalent serial cognate system lends solver-agnostic improvements over existing inverse dynamics methods for closed chains, by consolidating the RCM constraints and unactuated sublink inertial properties into the inertial properties of the cognate system.

### A. Computational Efficiency

There are many existing different approaches of modeling the loop closure constraint, such as cut joint and elastic joint modeling [12], but a common computational limitation even across formalisms is the need to model the rigid body equations for every link. The number of links N is certainly larger than the DoF O for these RCM systems. Thus, the mass matrix of the system is OxO whereas the joint space is N×1. Additional constraints must be introduced to produce a solvable system of equations either by Lagrange multipliers or virtual constraint forces for RNE methods. The inherent inefficiency in these strategies is the implicit modeling of constraint forces within the RCM which are not necessary for computing the inverse dynamics at the actuated joints. For RNE in particular, a minimum of 150N-48 multiplications and 131N-48 additions [1] would be required just for modeling the rigid body dynamics, and the constraint formulation can increase the number of operations substantially.

The proposed conversion to the serial cognate system reduces the number of links down to N′═O and yields a system where the number of links equals the DoF. In the isolated RCM case example from FIG. 4, N—O=6−2=4 rigid bodies corresponding to sublinks no longer need to be simulated. The elimination of passive sublinks and constraint forces within the RCM improves the computational efficiency at minimum to 150N′-48 multiplications and 131N′-48 additions. Additionally, the need to compute any constraint forces is completely removed. The precise number of operations removed depends on the type of constraint formulation originally used, but the reduction in operations from number of rigid bodies modeled is already considerable.

### V. Discussion

The method of creating equivalent serial links of closed chain RCM linkages and the results from the case study provide some insights into the potential applications, limitations, and future work on this topic.

The results from the case study demonstrate that the proposed method can accurately match the wrench of the original system to the serial cognate (FIG. 6), without sacrificing computational speed.

The requisite assumptions detailed in Section II-A present a few key limitations of the proposed method. First, the most common application of the wrench is to use it z component as the torque experienced by an actuator at that joint. While this equivalence is true for serial chain joints, the joint torque of an RCM closed chain actuator notably differs from the z component of the wrench across the joint. When our method is used to calculate joint actuator torques, it will accurately compute the actuator torque for all joints proximal and distal to the RCM, but has an error in the actuator torque for the RCM under conditions of high angular velocity and acceleration of links that are proximal to the RCM. However, the actuator torque matches exactly under the quasi-static conditions of joints proximal to the RCM defined in Section II-D. The serial cognate link representation thus provides a good approximation for systems where low velocities, and/or CG distances to instant centers of the RCM exist. The most prevalent application areas for RCMs are in physical human-robot interaction (pHRI) for tasks such as rehabilitation, assistance, augmentation, and robot-assisted surgery. These motions are relatively slow and can be approximated as quasi static

$$( {\to }_{α} = {\to }_{ω} = 0 ) .$$

The proposed method also requires planar mechanisms with axes of rotation that are parallel with RCM. However, most RCM mechanisms are planar [7]-[11], with a few notable spherical exceptions [23]-[25]. Despite the above limitations, our analysis shows that serial cognate link substitution is useful for robotic systems which comprise a mix of RCM closed chain linkages and serial links in order to reduce development time of custom closed chain linkage equations.

The next step in the validation of this approach is the characterization of the actuator torque error in non-quasistatic conditions. The specific sensitivities of the system to design parameters such as linkage geometry and mass distribution may be analyzed using a DOE, and even used as guidance for linkage design to minimize the RCM actuator torque error. Further, experimental characterization of the method in the real-time control of RCM mechanisms, with a focus on the degradation of the performance as the quasistatic assumptions are less valid and with the introduction of unmodeled nonlinearities.

Additional exploration of case studies could be used identify further limitations and establish the performance of the method in the context of other computational approaches. Lastly, future work should include extending this approach to non-planar mechanisms such as spherical RCMs.

### VI. Conclusion

We presented a method to replicate the inverse dynamics of RCM closed chain linkages as serial cognate links via a strategy of matching the CG Jacobian and inertia tensor of the two systems. The iteratively generated new cognate rotates about the RCMP of the RCM mechanism, and a mass position and inertia tensor are formed for this cognate link. The cognate link may then substituted for the RCM closed chain linkage in an RNE software package and DH parameters (or another kinematic representation) defined based on the RCM virtual center. The cognate wrench

$${\to }_{{F}_{i , cognate}} = f ( {}^{b} Θ , {}^{b} \dot{Θ} , {}^{b} \ddot{Θ} , {0}_{{\to }_{{v}_{b}}} , {0}_{{\to }_{{ω}_{b}}} , {0}_{{\to }_{{a}_{b}}} , {0}_{{\to }_{{α}_{b}}} , )$$

of the cognate link matches the wrench of the real RCM closed chain system perfectly, but the joint torque of the RCM closed chain actuator is only matched in the quasi-static case. With these constraints, the method stands to improve the real-time computation of the inverse dynamics of RCM mechanisms popular in pHRI applications such as rehabilitation and robot-assisted surgery.

### Nomenclature

{i} Reference frames at actuated joints
^{B}X^{C} Frame C rigid transform (pose) in frame B
^{ij}Iᵢⱼ Inertia matrix of sublink ij about frame ij
ⁱI′_{I} Inertia matrix of link i about frame i
Cᵢ Center of mass of link i, relative to frame i (only for RCM frames)
Cᵢⱼ Center of mass of sublink ij, relative to frame ij
ij Reference frame of a sub-link of an RCM
mᵢ mass of links associated with non-RCM frames
m′ᵢ Modified link masses (now all associated with serial links)
mᵢⱼ mass of sub-links associated with RCM frames
N Total number of rigid bodies in the original system n Number of actuated joints
N′ Total number of rigid bodies in serial cognate system
Nᵢ Number of rigid bodies associated with frame i

### REFERENCES

[1] J. M. Hollerbach, “A recursive Lagrangian formulation of manipulator dynamics and a comparative study of dynamics formulation complexity,” IEEE Trans. on Systems, Man, and Cybernetics, vol. SMC-10, 1980.
[2] W. M. Silver, “On the equivalence of Lagrangian and Newton-Euler dynamics for manipulators,” The Intl. J. of Robotics Res., vol. 1, 1982.
[3] PickNik Robotics. MoveIt. [Online]. Available: https://github.com/rosplanning/moveit2.
[4] The Math Works, Inc. MATLAB Robotics System Toolbox. [Online]. Available: https://www.mathworks.com/products/robotics.html.
[5] M. L. Felis, “RBDL: an efficient rigid-body dynamics library using recursive algorithms,” Autonomous Robots, pp. 1-17, 2016. [Online]. Available: http://dx.doi.org/10.1007/s10514-016-9574-0.
[6] Harmonic Bionics, Inc. Harmony SHR. [Online]. Available: https://www.harmonicbionics.com/harmony-shr/overview.
[7] B. Kim and A. D. Deshpande, “An upper-body rehabilitation exoskeleton Harmony with an anatomical shoulder mechanism: Design, modeling, control, and performance evaluation,” Intl. J. of Robotics Research, vol. 36, 2017.
[8] R. C. O. Locke and R. V. Patel, “Optimal remote center-of-motion location for robotics-assisted minimally-invasive surgery,” in Proceedings 2007 IEEE International Conference on Robotics and Automation, 2007, pp. 1900-1905.
[9] S. Aksungur, “Remote center of motion (RCM) mechanisms for surgical operations,” International Journal of Applied Mathematics, Electronics and Computers, vol. 3, 2015.
[10] H. M. Yip, P. Li, D. Navarro-Alarcon, Z. Wang, and Y. h. Liu, “A new circular-guided remote center of motion mechanism for assistive surgical robots,” in 2014 IEEE International Conference on Robotics and Biomimetics (ROBIO 2014), 2014, pp. 217-222.
[11] B. Eldridge, K. Gruben, D. LaRose, J. Funda, S. Gomory, J. Karidis, G. McVicker, R. Taylor, and J. Anderson, “A remote center of motion robotic arm for computer assisted surgery,” Robotica, vol. 14, 1996.
[12] F. Marques, I. Roupa, M. T. Silva, P. Flores, and H. M. Lankarani, “Examination and comparison of different methods to model closed loop kinematic chains using Lagrangian formulation with cut joint, clearance joint constraint and elastic joint approaches,” Mechanism and Machine Theory, vol. 160, 2021.
[13] B. Dasgupta and T. S. Mruthyunjaya, “A Newton-Euler formulation for the inverse dynamics of the stewart platform manipulator,” Mechanism and Machine Theory, vol. 33, 1998.
[14] Y. Li and Q. Xu, “Kinematics and inverse dynamics analysis for a general 3-PRS spatial parallel mechanism,” Robotica, vol. 23, 2005.
[15] C. M. Pappalardo, “A natural absolute coordinate formulation for the kinematic and dynamic analysis of rigid multibody systems,” Nonlinear Dynamics, vol. 81, 2015.
[16] X. M. Xu, J. H. Luo, X. G. Feng, H. J. Peng, and Z. G. Wu, “A generalized inertia representation for rigid multibody systems in terms of natural coordinates,” Mechanism and Machine Theory, vol. 157, 2021.
[17] H. Abdellatif and B. Heimann, “Computational efficient inverse dynamics of 6-DOF fully parallel manipulators by using the Lagrangian formalism,” Mechanism and Machine Theory, vol. 44, 2009.
[18] J. J. Uicker, “Dynamic force analysis of spatial linkages,” Journal of Applied Mechanics, Transactions ASME, vol. 34, 1964.
[19] M. Renaud, “Quasi-minimal computation of the dynamic model of a robot manipulator utilizing the Newton-Euler formalism and the notion of augmented body,” in Proceedings. International Conference on Robotics and Automation, vol. 4. IEEE, 1987, pp. 1677-1682.
[20] T. Sonoda, K. Ishii, and D. Isobe, “Dynamics computation of link mechanisms employing COG Jacobian,” in IEEE/ASME International Conference on Advanced Intelligent Mechatronics, AIM. IEEE, 2008, pp. 482-487.
[21] K. M. Lynch and F. C. Park, Modern Robotics: Mechanics, Planning, and Control. Cambridge University Press, 2017, vol. 48.
[22] K. Liu and S. Jain, A Quick Tutorial on Multibody Dynamics, School of Interactive Computing, Georgia Institute of Technology.
[23] S. J. Harris, F. Arambula-Cosio, and Q. Mei, “The Probot—an active robot for prostate resection,” Proceedings of the Institution of Mechanical Engineers, Part H: Journal of Engineering in Medicine, vol. 211, 1997.
[24] K. Masamune, E. Kobayashi, Y. Masutani, M. Suzuki, T. Dohi, H. Iseki, and K. Takakura, “Development of an MRI-compatible needle insertion manipulator for stereotactic neurosurgery,” Computer Aided Surgery, vol. 1, 1995.
[25] D. Kim, E. Kobayashi, T. Dohi, and I. Sakuma, “A new, compact MR-compatible surgical manipulator for minimally invasive liver surgery,” in Medical Image Computing and Computer-Assisted Intervention—MICCAI 2002: 5th International Conference_Tokyo, Japan, Sep. 25-28, 2002 Proceedings, Part I 5. Springer, 2002, pp. 99-106.

### Example Set 1

Example 1. A system comprising: a closed chain robotic linkage (FIG. 13, left linkage); at least one memory; at least one processor, coupled to the at least one memory and the closed chain robotic linkage, to perform operations comprising: representing the closed chain robotic linkage as a serial chain robotic linkage (FIG. 13, right linkage); using a Recursive Newton Euler (RNE) package on the serial chain robotic linkage to determine real-time inverse dynamics related to the serial chain robotic linkage; in response to using the RNE package on the serial chain robotic linkage to determine real-time inverse dynamics related to the serial chain robotic linkage, determining real-time inverse dynamics related to the closed chain robotic linkage; in response to determining the real-time inverse dynamics related to the closed chain robotic linkage, exercising real time control of the closed chain robotic linkage to physically move the closed chain robotic linkage from a first physical position to a second physical position.

For example, based on the cognate system model, the real world physical linkage may be rotated such as with a robot forearm joint rotating about a RCM.

Example 1. (Alternative version) A system comprising: a closed chain robotic linkage; at least one memory; at least one processor, coupled to the at least one memory and the closed chain robotic linkage, to perform operations comprising: representing the closed chain robotic linkage as a serial chain robotic linkage; using a serial chain solver on the serial chain robotic linkage to determine real-time inverse dynamics related to the serial chain robotic linkage; in response to using the serial chain solver on the serial chain robotic linkage to determine real-time inverse dynamics related to the serial chain robotic linkage, determining real-time inverse dynamics related to the closed chain robotic linkage; in response to determining the real-time inverse dynamics related to the closed chain robotic linkage, exercising real time control of the closed chain robotic linkage to physically move the closed chain robotic linkage from a first physical position to a second physical position.

For example, a serial chain solver may use RNE techniques, Lagrangian techniques, and the like.

Example 2. The system of example 1, wherein: the closed chain robotic linkage comprises: (1) a remote center of motion (RCM), (2) a closed chain robotic linkage of sublinks (Sublinks 31, 32, 33, 34), and (3) an additional link (Link 3) coupled to the closed chain robotic linkage of sublinks; and the additional link is constrained to rotate about a fixed remote center of motion point (RCMP) (FIG. 13, CM Point).

For example, in FIG. 13 Link 2 is an input link and Link 3 is an output link. The closed chain robotic linkage of sublinks do not rotate about the RCMP.

Example 3. The system of example 2, wherein the closed chain robotic linkage of sublinks constrain the additional link such that the additional link is to behave as a serial link.

Example 4. The system according to any of examples 2-3, wherein the representing the closed chain robotic linkage as the serial chain robotic linkage includes determining equivalency statements for inverse dynamics between the closed chain robotic linkage of sublinks and both of the serial chain robotic first link and the serial chain robotic second link.

Example 5. The system of example 4, wherein the representing the closed chain robotic linkage as the serial chain robotic linkage includes solving the equivalency statements to produce inertial properties for the serial chain robotic linkage.

Example 6. The system of example 5, wherein the determining equivalency statements for inverse dynamics between the closed chain robotic linkage of sublinks and both of the serial chain robotic first link and the serial chain robotic second link includes mapping masses of the closed chain robotic linkage of sublinks to masses of the serial chain robotic first link and the serial chain robotic second link.

Example 7. The system of example 6, wherein the determining equivalency statements for inverse dynamics between the closed chain robotic linkage of sublinks and both of the serial chain robotic first link and the serial chain robotic second link includes mapping inertias of the closed chain robotic linkage of sublinks to inertias of the serial chain robotic first link and the serial chain robotic second link.

Example 8. The system according to any of examples 2-7, wherein the representing the closed chain robotic linkage as the serial chain robotic linkage includes parameterizing kinematics for the closed chain robotic linkage of sublinks.

Example 9. The system according to any of examples 2-8, wherein the closed chain robotic linkage of sublinks includes more than two sublinks.

Example 10. The system of example 9, wherein at least two of the closed chain robotic linkage of sublinks are parallel to each other.

Example 11. The system of example 10, wherein at least two of the closed chain robotic linkage of sublinks have rotation axes parallel to each other.

Example 12. The system according to any of examples 2-11, wherein no physical joint exists at the RCMP.

Example 13. The system according to any of examples 2-12, wherein the representing the closed chain robotic linkage as the serial chain robotic linkage includes solving inertial-equivalency statements between the closed chain robotic linkage and the serial chain robotic linkage.

Example 14. The system according to any of examples 2-13, wherein: the serial chain robotic linkage comprises a serial chain robotic first link (Link 2′); the serial chain robotic linkage comprises a serial chain robotic second link (Link 3′); the serial chain robotic second link is constrained to rotate about the fixed RCMP.

Example 15. The system of example 14, wherein the serial chain robotic first link is coupled to the serial chain robotic second link at the fixed RCMP.

Example 16. The system of example 15, wherein the serial chain robotic second link is constrained to rotate about the fixed RCMP.

Example 17. The system according to any of examples 2-16, wherein the RCM is a 1-degree of freedom planar RCM.

Example 18. The system according to any of examples 2-17, wherein the closed chain robotic linkage is a physical linkage.

Example 19. The system of example 18, wherein the serial chain robotic linkage is a digital linkage.

Example 20. The system of example 18, wherein the serial chain robotic linkage is a simulated version of the closed chain robotic linkage.

Example 21. The system according to any examples 2-20, wherein the using the RNE package on the serial chain robotic linkage to determine real-time inverse dynamics related to the serial chain robotic linkage includes using the RNE package on the serial chain robotic linkage to determine real-time torques related to the serial chain robotic linkage.

Example 21 (Alternative version). The system according to any of examples 2-20, wherein the using the serial chain solver on the serial chain robotic linkage to determine real-time inverse dynamics related to the serial chain robotic linkage includes using the serial chain solver on the serial chain robotic linkage to determine real-time torques related to the serial chain robotic linkage.

Example 22. The system of example 21, wherein the using the RNE package on the serial chain robotic linkage to determine real-time inverse dynamics related to the serial chain robotic linkage includes using the RNE package on the serial chain robotic linkage to determine real-time Coriolis forces related to the serial chain robotic linkage.

Example 22 (alternative version). The system of example 21, wherein the using the serial chain solver on the serial chain robotic linkage to determine real-time inverse dynamics related to the serial chain robotic linkage includes using the serial chain solver on the serial chain robotic linkage to determine real-time Coriolis forces related to the serial chain robotic linkage.

Example 23. The system according to any of examples 2-22, wherein: the closed chain robotic linkage is included in a limb of an exoskeleton; the exercising real time control of the closed chain robotic linkage to physically move the closed chain robotic linkage from a first physical position to a second physical position includes exercising real time control of the limb to physically move the limb from a first physical position to a second physical position.

Example 24. The system of example 23, wherein the operations comprise performing physical therapy on a patient coupled to the exoskeleton by exercising real time control of the limb to physically move the limb from a first physical position to a second physical position.

Example 25. The system of example 24, wherein the patient includes a body part located at the RCMP.

Example 26. The system according to any of examples 2-25, wherein the closed chain robotic linkage includes a loop of links formed by the closed chain robotic linkage of sublinks.

Example 27. The system of example 26, wherein neither of the serial chain robotic first link nor the serial chain robotic second link is included in a loop of links.

Example 28. At least one machine readable medium comprising a plurality of instructions that in response to being executed on a computing device, cause the computing device to carry out the operations according to any one of examples 1 to 27.

Example 29. A method executed by at least one processor, the method including the operations according to any one of examples 1 to 27.

### Example Set 2

Example 1. A system comprising: a closed chain robotic linkage (FIG. 13, left linkage); at least one memory; at least one processor, coupled to the at least one memory and the closed chain robotic linkage, to perform operations comprising: representing the closed chain robotic linkage as a serial chain robotic linkage (FIG. 13, right linkage); using a Recursive Newton Euler (RNE) package on the serial chain robotic linkage to determine real-time inverse dynamics related to the serial chain robotic linkage; in response to using the RNE package on the serial chain robotic linkage to determine real-time inverse dynamics related to the serial chain robotic linkage, determining real-time inverse dynamics related to the closed chain robotic linkage; in response to determining the real-time inverse dynamics related to the closed chain robotic linkage, exercising real time control of the closed chain robotic linkage to physically move the closed chain robotic linkage from a first physical position to a second physical position.

For example, based on the cognate system model, the real world physical linkage may be rotated such as with a robot forearm joint rotating about a RCM.

Example 1. (Alternative version) A system comprising: a closed chain robotic linkage; at least one memory; at least one processor, coupled to the at least one memory and the closed chain robotic linkage, to perform operations comprising: representing the closed chain robotic linkage as a serial chain robotic linkage; using a serial chain solver on the serial chain robotic linkage to determine real-time inverse dynamics related to the serial chain robotic linkage; in response to using the serial chain solver on the serial chain robotic linkage to determine real-time inverse dynamics related to the serial chain robotic linkage, determining real-time inverse dynamics related to the closed chain robotic linkage; in response to determining the real-time inverse dynamics related to the closed chain robotic linkage, exercising real time control of the closed chain robotic linkage to physically move the closed chain robotic linkage from a first physical position to a second physical position.

Example 2. The system of example 1, wherein: the closed chain robotic linkage comprises: (1) a remote center of motion (RCM), (2) a closed chain robotic linkage of sublinks (Sublinks 31, 32, 33, 34), and (3) an additional link (Link 3) coupled to the closed chain robotic linkage of sublinks; and the additional link is constrained to rotate about a fixed remote center of motion point (RCMP) (FIG. 13, CM Point).

Example 3. The system of example 2, wherein the closed chain robotic linkage of sublinks constrain the additional link such that the additional link is to behave as a serial link.

Example 4. The system of example 2, wherein the representing the closed chain robotic linkage as the serial chain robotic linkage includes determining equivalency statements for inverse dynamics between the closed chain robotic linkage of sublinks and both of the serial chain robotic first link and the serial chain robotic second link.

Example 5. The system of example 4, wherein the representing the closed chain robotic linkage as the serial chain robotic linkage includes solving the equivalency statements to produce inertial properties for the serial chain robotic linkage.

Example 6. The system of example 5, wherein the determining equivalency statements for inverse dynamics between the closed chain robotic linkage of sublinks and both of the serial chain robotic first link and the serial chain robotic second link includes mapping masses of the closed chain robotic linkage of sublinks to masses of the serial chain robotic first link and the serial chain robotic second link.

Example 7. The system of example 6, wherein the determining equivalency statements for inverse dynamics between the closed chain robotic linkage of sublinks and both of the serial chain robotic first link and the serial chain robotic second link includes mapping inertias of the closed chain robotic linkage of sublinks to inertias of the serial chain robotic first link and the serial chain robotic second link.

Example 8. The system of example 2, wherein the representing the closed chain robotic linkage as the serial chain robotic linkage includes parameterizing kinematics for the closed chain robotic linkage of sublinks.

Example 9. The system of example 2, wherein the closed chain robotic linkage of sublinks includes more than two sublinks.

Example 10. The system of example 9, wherein at least two of the closed chain robotic linkage of sublinks are parallel to each other.

Example 11. The system of example 10, wherein at least two of the closed chain robotic linkage of sublinks have rotation axes parallel to each other.

Example 12. The system of example 2, wherein no physical joint exists at the RCMP.

Example 13. The system of example 2, wherein the representing the closed chain robotic linkage as the serial chain robotic linkage includes solving inertial-equivalency statements between the closed chain robotic linkage and the serial chain robotic linkage.

Example 14. The system of example 2, wherein: the serial chain robotic linkage comprises a serial chain robotic first link (Link 2′); the serial chain robotic linkage comprises a serial chain robotic second link (Link 3′); the serial chain robotic second link is constrained to rotate about the fixed RCMP.

Example 15. The system of example 14, wherein the serial chain robotic first link is coupled to the serial chain robotic second link at the fixed RCMP.

Example 16. The system of example 15, wherein the serial chain robotic second link is constrained to rotate about the fixed RCMP.

Example 17. The system of example 2, wherein the RCM is a 1-degree of freedom planar RCM.

Example 18. The system of example 2, wherein the closed chain robotic linkage is a physical linkage.

Example 19. The system of example 18, wherein the serial chain robotic linkage is a digital linkage.

Example 20. The system of example 18, wherein the serial chain robotic linkage is a simulated version of the closed chain robotic linkage.

Example 21. The system of example 2, wherein the using the RNE package on the serial chain robotic linkage to determine real-time inverse dynamics related to the serial chain robotic linkage includes using the RNE package on the serial chain robotic linkage to determine real-time torques related to the serial chain robotic linkage.

Example 21 (Alternative version). The system of example 2, wherein the using the serial chain solver on the serial chain robotic linkage to determine real-time inverse dynamics related to the serial chain robotic linkage includes using the serial chain solver on the serial chain robotic linkage to determine real-time torques related to the serial chain robotic linkage.

Example 22. The system of example 21, wherein the using the RNE package on the serial chain robotic linkage to determine real-time inverse dynamics related to the serial chain robotic linkage includes using the RNE package on the serial chain robotic linkage to determine real-time Coriolis forces related to the serial chain robotic linkage.

Example 22 (alternative version). The system of example 21, wherein the using the serial chain solver on the serial chain robotic linkage to determine real-time inverse dynamics related to the serial chain robotic linkage includes using the serial chain solver on the serial chain robotic linkage to determine real-time Coriolis forces related to the serial chain robotic linkage.

Example 23. The system of example 2, wherein: the closed chain robotic linkage is included in a limb of an exoskeleton; the exercising real time control of the closed chain robotic linkage to physically move the closed chain robotic linkage from a first physical position to a second physical position includes exercising real time control of the limb to physically move the limb from a first physical position to a second physical position.

Example 24. The system of example 23, wherein the operations comprise performing physical therapy on a patient coupled to the exoskeleton by exercising real time control of the limb to physically move the limb from a first physical position to a second physical position.

Example 25. The system of example 24, wherein the patient includes a body part located at the RCMP.

Example 26. The system of example 2, wherein the closed chain robotic linkage includes a loop of links formed by the closed chain robotic linkage of sublinks.

Example 27. The system of example 26, wherein neither of the serial chain robotic first link nor the serial chain robotic second link is included in a loop of links.

Example 28. At least one machine readable medium comprising a plurality of instructions that in response to being executed on a computing device, cause the computing device to carry out the operations according to any one of examples 1 to 27.

Example 29. A method executed by at least one processor, the method including the operations according to any one of examples 1 to 27.

### Example Set 3

Example 1. A method of decreasing latency in a computer-based robotic physical rehabilitation system, the method comprising: representing a closed chain robotic linkage as a serial chain robotic linkage; using a serial chain solver on the serial chain robotic linkage to determine real-time inverse dynamics related to the serial chain robotic linkage; in response to using the serial chain solver on the serial chain robotic linkage to determine real-time inverse dynamics related to the serial chain robotic linkage, determining real-time inverse dynamics related to the closed chain robotic linkage; in response to determining the real-time inverse dynamics related to the closed chain robotic linkage, exercising real time control of the closed chain robotic linkage to physically move the closed chain robotic linkage from a first physical position to a second physical position.

Example 2. The method of example 1, wherein: the closed chain robotic linkage comprises: (1) a remote center of motion (RCM), (2) a closed chain robotic linkage of sublinks (Sublinks 31, 32, 33, 34), and (3) an additional link (Link 3) coupled to the closed chain robotic linkage of sublinks; and the additional link is constrained to rotate about a fixed remote center of motion point (RCMP) (FIG. 13, CM Point).

Example 3. The method of example 2, wherein the closed chain robotic linkage of sublinks constrain the additional link such that the additional link is to behave as a serial link.

Example 4. The method of example 2, wherein the representing the closed chain robotic linkage as the serial chain robotic linkage includes determining equivalency statements for inverse dynamics between the closed chain robotic linkage of sublinks and both of the serial chain robotic first link and the serial chain robotic second link.

Example 5. The method of example 4, wherein the representing the closed chain robotic linkage as the serial chain robotic linkage includes solving the equivalency statements to produce inertial properties for the serial chain robotic linkage.

Example 6. The method of example 5, wherein the determining equivalency statements for inverse dynamics between the closed chain robotic linkage of sublinks and both of the serial chain robotic first link and the serial chain robotic second link includes mapping masses of the closed chain robotic linkage of sublinks to masses of the serial chain robotic first link and the serial chain robotic second link.

Example 7. The method of example 6, wherein the determining equivalency statements for inverse dynamics between the closed chain robotic linkage of sublinks and both of the serial chain robotic first link and the serial chain robotic second link includes mapping inertias of the closed chain robotic linkage of sublinks to inertias of the serial chain robotic first link and the serial chain robotic second link.

Example 8. The method of example 2, wherein the representing the closed chain robotic linkage as the serial chain robotic linkage includes parameterizing kinematics for the closed chain robotic linkage of sublinks.

Example 9. The method of example 2, wherein the closed chain robotic linkage of sublinks includes more than two sublinks.

Example 10. The method of example 9, wherein at least two of the closed chain robotic linkage of sublinks are parallel to each other.

Example 11. The method of example 10, wherein at least two of the closed chain robotic linkage of sublinks have rotation axes parallel to each other.

Example 12. The method of example 2, wherein no physical joint exists at the RCMP.

Example 13. The method of example 2, wherein the representing the closed chain robotic linkage as the serial chain robotic linkage includes solving inertial-equivalency statements between the closed chain robotic linkage and the serial chain robotic linkage.

Example 14. The method of example 2, wherein: the serial chain robotic linkage comprises a serial chain robotic first link (Link 2′); the serial chain robotic linkage comprises a serial chain robotic second link (Link 3′); the serial chain robotic second link is constrained to rotate about the fixed RCMP.

Example 15. The method of example 14, wherein the serial chain robotic first link is coupled to the serial chain robotic second link at the fixed RCMP.

Example 16. The method of example 15, wherein the serial chain robotic second link is constrained to rotate about the fixed RCMP.

Example 17. The method of example 2, wherein the RCM is a 1-degree of freedom planar RCM.

Example 18. The method of example 2, wherein the closed chain robotic linkage is a physical linkage.

Example 19. The method of example 18, wherein the serial chain robotic linkage is a digital linkage.

Example 20. The method of example 18, wherein the serial chain robotic linkage is a simulated version of the closed chain robotic linkage.

Example 21. The method of example 2, wherein the using the RNE package on the serial chain robotic linkage to determine real-time inverse dynamics related to the serial chain robotic linkage includes using the RNE package on the serial chain robotic linkage to determine real-time torques related to the serial chain robotic linkage.

Example 21 (Alternative version). The method of example 2, wherein the using the serial chain solver on the serial chain robotic linkage to determine real-time inverse dynamics related to the serial chain robotic linkage includes using the serial chain solver on the serial chain robotic linkage to determine real-time torques related to the serial chain robotic linkage.

Example 22. The method of example 21, wherein the using the RNE package on the serial chain robotic linkage to determine real-time inverse dynamics related to the serial chain robotic linkage includes using the RNE package on the serial chain robotic linkage to determine real-time Coriolis forces related to the serial chain robotic linkage.

Example 22 (alternative version). The method of example 21, wherein the using the serial chain solver on the serial chain robotic linkage to determine real-time inverse dynamics related to the serial chain robotic linkage includes using the serial chain solver on the serial chain robotic linkage to determine real-time Coriolis forces related to the serial chain robotic linkage.

Example 23. The method of example 2, wherein: the closed chain robotic linkage is included in a limb of an exoskeleton; the exercising real time control of the closed chain robotic linkage to physically move the closed chain robotic linkage from a first physical position to a second physical position includes exercising real time control of the limb to physically move the limb from a first physical position to a second physical position.

Example 24. The method of example 23, wherein the operations comprise performing physical therapy on a patient coupled to the exoskeleton by exercising real time control of the limb to physically move the limb from a first physical position to a second physical position.

Example 25. The method of example 24, wherein the patient includes a body part located at the RCMP.

Example 26. The method of example 2, wherein the closed chain robotic linkage includes a loop of links formed by the closed chain robotic linkage of sublinks.

Example 27. The method of example 26, wherein neither of the serial chain robotic first link nor the serial chain robotic second link is included in a loop of links.

### Example Set 4

Example 1. A method comprising: representing a closed chain robotic linkage as a serial chain robotic linkage; using a serial chain solver on the serial chain robotic linkage to determine real-time inverse dynamics related to the serial chain robotic linkage; in response to using the serial chain solver on the serial chain robotic linkage to determine real-time inverse dynamics related to the serial chain robotic linkage, determining real-time inverse dynamics related to the closed chain robotic linkage.

For example, an embodiment may be directed towards simulation software.

Example 2. The method of example 1, wherein: the closed chain robotic linkage comprises: (1) a remote center of motion (RCM), (2) a closed chain robotic linkage of sublinks (Sublinks 31, 32, 33, 34), and (3) an additional link (Link 3) coupled to the closed chain robotic linkage of sublinks; and the additional link is constrained to rotate about a fixed remote center of motion point (RCMP) (FIG. 13, CM Point).

Example 3. The method of example 2, wherein the closed chain robotic linkage of sublinks constrain the additional link such that the additional link is to behave as a serial link.

Example 4. The method of example 2, wherein the representing the closed chain robotic linkage as the serial chain robotic linkage includes determining equivalency statements for inverse dynamics between the closed chain robotic linkage of sublinks and both of the serial chain robotic first link and the serial chain robotic second link.

Example 5. The method of example 4, wherein the representing the closed chain robotic linkage as the serial chain robotic linkage includes solving the equivalency statements to produce inertial properties for the serial chain robotic linkage.

Example 6. The method of example 5, wherein the determining equivalency statements for inverse dynamics between the closed chain robotic linkage of sublinks and both of the serial chain robotic first link and the serial chain robotic second link includes mapping masses of the closed chain robotic linkage of sublinks to masses of the serial chain robotic first link and the serial chain robotic second link.

Example 7. The method of example 6, wherein the determining equivalency statements for inverse dynamics between the closed chain robotic linkage of sublinks and both of the serial chain robotic first link and the serial chain robotic second link includes mapping inertias of the closed chain robotic linkage of sublinks to inertias of the serial chain robotic first link and the serial chain robotic second link.

Example 8. The method of example 2, wherein the representing the closed chain robotic linkage as the serial chain robotic linkage includes parameterizing kinematics for the closed chain robotic linkage of sublinks.

Example 9. The method of example 2, wherein the closed chain robotic linkage of sublinks includes more than two sublinks.

Example 10. The method of example 9, wherein at least two of the closed chain robotic linkage of sublinks are parallel to each other.

Example 11. The method of example 10, wherein at least two of the closed chain robotic linkage of sublinks have rotation axes parallel to each other.

Example 12. The method of example 2, wherein no physical joint exists at the RCMP.

Example 13. The method of example 2, wherein the representing the closed chain robotic linkage as the serial chain robotic linkage includes solving inertial-equivalency statements between the closed chain robotic linkage and the serial chain robotic linkage.

Example 14. The method of example 2, wherein: the serial chain robotic linkage comprises a serial chain robotic first link (Link 2′); the serial chain robotic linkage comprises a serial chain robotic second link (Link 3′); the serial chain robotic second link is constrained to rotate about the fixed RCMP.

Example 15. The method of example 14, wherein the serial chain robotic first link is coupled to the serial chain robotic second link at the fixed RCMP.

Example 16. The method of example 15, wherein the serial chain robotic second link is constrained to rotate about the fixed RCMP.

Example 17. The method of example 2, wherein the RCM is a 1-degree of freedom planar RCM.

Example 18. The method of example 2, wherein the closed chain robotic linkage is a physical linkage.

Example 19. The method of example 18, wherein the serial chain robotic linkage is a digital linkage.

Example 20. The method of example 18, wherein the serial chain robotic linkage is a simulated version of the closed chain robotic linkage.

Example 21. The method of example 2, wherein the using the RNE package on the serial chain robotic linkage to determine real-time inverse dynamics related to the serial chain robotic linkage includes using the RNE package on the serial chain robotic linkage to determine real-time torques related to the serial chain robotic linkage.

Example 21 (Alternative version). The method of example 2, wherein the using the serial chain solver on the serial chain robotic linkage to determine real-time inverse dynamics related to the serial chain robotic linkage includes using the serial chain solver on the serial chain robotic linkage to determine real-time torques related to the serial chain robotic linkage.

Example 22. The method of example 21, wherein the using the RNE package on the serial chain robotic linkage to determine real-time inverse dynamics related to the serial chain robotic linkage includes using the RNE package on the serial chain robotic linkage to determine real-time Coriolis forces related to the serial chain robotic linkage.

Example 22 (alternative version). The method of example 21, wherein the using the serial chain solver on the serial chain robotic linkage to determine real-time inverse dynamics related to the serial chain robotic linkage includes using the serial chain solver on the serial chain robotic linkage to determine real-time Coriolis forces related to the serial chain robotic linkage.

Example 23. The method of example 2, wherein the closed chain robotic linkage includes a loop of links formed by the closed chain robotic linkage of sublinks.

Example 24. The method of example 23, wherein neither of the serial chain robotic first link nor the serial chain robotic second link is included in a loop of links.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A system comprising:
a closed chain robotic linkage;
at least one memory;
at least one processor, coupled to the at least one memory and the closed chain robotic linkage, to perform operations comprising:
representing the closed chain robotic linkage as a serial chain robotic linkage;
using a serial chain solver on the serial chain robotic linkage to determine real-time inverse dynamics related to the serial chain robotic linkage;
in response to using the serial chain solver on the serial chain robotic linkage to determine real-time inverse dynamics related to the serial chain robotic linkage, determining real-time inverse dynamics related to the closed chain robotic linkage;
in response to determining the real-time inverse dynamics related to the closed chain robotic linkage, exercising real time control of the closed chain robotic linkage to physically move the closed chain robotic linkage from a first physical position to a second physical position.

2. The system of claim 1, wherein:
the closed chain robotic linkage comprises: (1) a remote center of motion (RCM), (2) a closed chain robotic linkage of sublinks, and (3) an additional link coupled to the closed chain robotic linkage of sublinks; and
the additional link is constrained to rotate about a fixed remote center of motion point (RCMP).

3. The system of claim 2, wherein the closed chain robotic linkage of sublinks constrain the additional link such that the additional link is to behave as a serial link.

4. The system according to claim 3, wherein the representing the closed chain robotic linkage as the serial chain robotic linkage includes determining equivalency statements for inverse dynamics between the closed chain robotic linkage of sublinks and both of a serial chain robotic first link and a serial chain robotic second link.

5. The system of claim 4, wherein the representing the closed chain robotic linkage as the serial chain robotic linkage includes solving the equivalency statements to produce inertial properties for the serial chain robotic linkage.

6. The system of claim 5, wherein the determining equivalency statements for inverse dynamics between the closed chain robotic linkage of sublinks and both of the serial chain robotic first link and the serial chain robotic second link includes mapping masses of the closed chain robotic linkage of sublinks to masses of the serial chain robotic first link and the serial chain robotic second link.

7. The system of claim 6, wherein the determining equivalency statements for inverse dynamics between the closed chain robotic linkage of sublinks and both of the serial chain robotic first link and the serial chain robotic second link includes mapping inertias of the closed chain robotic linkage of sublinks to inertias of the serial chain robotic first link and the serial chain robotic second link.

8. The system according to claim 7, wherein the representing the closed chain robotic linkage as the serial chain robotic linkage includes parameterizing kinematics for the closed chain robotic linkage of sublinks.

9. The system according to claim 8, wherein the closed chain robotic linkage of sublinks includes more than two sublinks.

10. The system of claim 9, wherein at least two of the closed chain robotic linkage of sublinks are parallel to each other.

11. The system of claim 10, wherein at least two of the closed chain robotic linkage of sublinks have rotation axes parallel to each other.

12. The system according to claim 11, wherein no physical joint exists at the RCMP.

13. The system according to claim 12, wherein the representing the closed chain robotic linkage as the serial chain robotic linkage includes solving inertial-equivalency statements between the closed chain robotic linkage and the serial chain robotic linkage.

14. The system according to claim 2, wherein:
the serial chain robotic linkage comprises a serial chain robotic first link (Link 2′);
the serial chain robotic linkage comprises a serial chain robotic second link (Link 3′);
the serial chain robotic second link is constrained to rotate about the fixed RCMP.

15. The system of claim 14, wherein the serial chain robotic first link is coupled to the serial chain robotic second link at the fixed RCMP.

16. The system of claim 15, wherein the serial chain robotic second link is constrained to rotate about the fixed RCMP.

17. The system according to claim 16, wherein the RCM is a 1-degree of freedom planar RCM.

18. The system according to claim 17, wherein the closed chain robotic linkage is a physical linkage.

19. The system of claim 18, wherein the serial chain robotic linkage is a digital linkage.

20. The system of claim 18, wherein the serial chain robotic linkage is a simulated version of the closed chain robotic linkage.

21. The system according to claim 20, wherein the using the serial chain solver on the serial chain robotic linkage to determine real-time inverse dynamics related to the serial chain robotic linkage includes using the serial chain solver on the serial chain robotic linkage to determine real-time torques related to the serial chain robotic linkage.

22. The system of claim 21, wherein the using the serial chain solver on the serial chain robotic linkage to determine real-time inverse dynamics related to the serial chain robotic linkage includes using the serial chain solver on the serial chain robotic linkage to determine real-time Coriolis forces related to the serial chain robotic linkage.

23. The system according to claim 22, wherein:
the closed chain robotic linkage is included in a limb of an exoskeleton;
the exercising real time control of the closed chain robotic linkage to physically move the closed chain robotic linkage from a first physical position to a second physical position includes exercising real time control of the limb to physically move the limb from a first physical position to a second physical position.

24. The system of claim 23, wherein the operations comprise performing physical therapy on a patient coupled to the exoskeleton by exercising real time control of the limb to physically move the limb from the first physical position to the second physical position.

25. The system of claim 24, wherein the patient includes a body part located at the RCMP.

26. The system according to claim 25, wherein the closed chain robotic linkage includes a loop of links formed by the closed chain robotic linkage of sublinks.

27. The system of claim 26, wherein neither of the serial chain robotic first link nor the serial chain robotic second link is included in a loop of links.

28.-29. (canceled)
